(19) United States
(12) Patent Application Publication
(10) Pub. No.: US 2026/0070881 A1
(43) Pub. Date: Mar. 12, 2026

(54) Photochromic Indeno-Fused Naphthopyran Compounds, Compositions, and Articles Containing Same

(71) Applicant: Transitions Optical, Ltd., Tuam (IE)

(72) Inventors: Wenjing Xiao, Murrysville, PA (US); Patrick M. Brown, McKees Rocks, PA (US); Chien-Yang Chiu, Export, PA (US); Robert W. Walters, Murrysville, PA (US)

(21) Appl. No.: 19/106,256

(22) PCT Filed: Aug. 30, 2022
(86) PCT No.: PCT/EP2022/074001

§ 371 Date: Feb. 25, 2025

(51) Int. Cl. C07D 311/94; C07D 405/04; C09K 9/02; G02B 5/23; G02C 7/10

(52) U.S. Cl. CPC C07D 311/94; C07D 405/04; C09K 9/02; G02B 5/23; G02C 7/102; C09K 2211/1007; C09K 2211/1011; C09K 2211/1018

(57) ABSTRACT

An indeno-fused naphthopyran having the core skeletal structure (I): (I) wherein R¹ is a substituted or unsubstituted alkyl group; R² is substituted or unsubstituted amino; substituted or unsubstituted alkyl; substituted or unsubstituted alkenyl; substituted or unsubstituted alkynyl; substituted or unsubstituted aryl; substituted or unsubstituted heteroaryl; substituted or unsubstituted alkoxy; substituted or unsubstituted aryloxy; substituted or unsubstituted alkylthio; substituted or unsubstituted aryl thio; or substituted or unsubstituted amide, urea, and carbamate; and at least one of R³ and R⁴ is an electron donating group, wherein the sum of the Hammett σₚ values for R³ and R⁴ is less than −0.40, wherein when R₁ is a substituted alkyl group, a carbon atom of the alkyl group is directly bonded to the 7-position carbon atom, and the carbon atom of the alkyl group that is directly bonded to the 7-position carbon atom is unsubstituted.

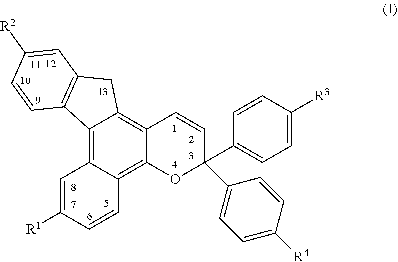

### FIELD

The present invention relates to fused ring photochromic compounds, such as photochromic indeno-fused naphthopyran compounds, and photochromic compositions and photochromic articles that include such photochromic compounds.

### BACKGROUND

Photochromic compounds undergo a transformation from one state (or form) to another state in response to certain wavelengths of electromagnetic radiation (i.e., “actinic radiation”). Each state has a characteristic absorption spectrum. For example, many photochromic compounds transform from an unactivated (e.g., bleached or substantially colorless) state to an activated (e.g., colored or tinted) state upon exposure to actinic radiation. When the actinic radiation is removed, the photochromic compounds reversibly transform from the activated state back to the bleached, unactivated state. A “thermally reversible photochromic compound” is a photochromic compound that converts from an unactivated state to an activated state in response to actinic radiation, and reverts back to the unactivated state in response to thermal energy. The activation reaction (from unactivated to activated) is primarily photochemical, while the deactivation reaction (from activated to unactivated) is primarily thermal.

Known photochromic compounds, such as photochromic indeno-fused naphthopyran compounds exhibit the advantages of good darkness in the activated state, as well as acceptable fade rate when transitioning back to the bleached, unactivated state. In some instances, however, these photochromic compounds can exhibit undesirable tint or color in the bleached, unactivated state. This generally is referred to as “bleach color.” This bleach color can negatively impact the aesthetics of articles, such as optical articles, which comprise such photochromic compounds.

Photochromic ophthalmic lenses offer the lens wearer glare reduction upon exposure to solar (actinic) radiation which prompts rapid coloration of the lens, thereby reducing the amount of light reaching the wearer's eyes. Upon return to an indoor environment, any lens coloration or tint remaining in the lens can be detrimental to the wearer from both a cosmetic and a functional perspective. Therefore, it is desirable to provide a photochromatic lens that is clear when the lens wearer is indoors and colored/tinted, serving as a sun lens, while the wearer is outdoors. This can be accomplished through the use of photochromic dyes that provide high absorptivity in the activated state, with rapid speeds for switching from the clear (unactivated, indoor) state to the colored (darkened, outdoor) state, and back again. Photochromic dyes that have minimal residual color or tint when the wearer is indoors (i.e., in the unactivated state) are necessary to yield the highest quality photochromic lenses.

Further, combinations of photochromic compounds with different activated colors are often used to produce photochromic lenses that are neutral grey or brown upon activation. For example, mixtures of photochromic compounds with blue and orange activated states can be combined to produce a neutral grey color. It is desirable to have the activation and fade rates of these different compounds to be as similar as possible to maintain color uniformity during both activation and fade. For these reasons, it is necessary that compounds which are blue in the activated state have high absorptivity in the activated state, have fast fade rates, and minimal color in the unactivated (clear) state.

Compounds described in U.S. Pat. No. 9,028,728, for example, can exhibit desirable levels of blue coloration in the activated state but can suffer from residual color in the bleach state. This residual tint can manifest as yellowness (b*) due to the absorption of the closed form of the photochromic dye into high energy, visible region of the solar spectrum. The residual tint can also be a result of thermochromism of the dyes that leads to low levels of the photochromic dyes remaining in the activated state while indoors and not fully returning to the unactivated state. This is often measured as a decrease in the unactivated transmittance of the sample.

Further, known photochromic indeno-fused naphthopyran compounds having no substitution at the 7-position and strong electron donor groups on the 3,3′-aryl groups can provide good bleach color but can suffer from a less dark activated state. The lessened activated outdoor darkness of such compounds, especially at warm temperatures, can be a limitation of photochromic lenses containing such compounds.

Delta E_{% T} represents the percent transmission (% T), the a* values, and b* values of an unactivated photochromic layer to that of the transparent substrate (% Tₒ, a*₀, b*₀) to which the photochromic layer is applied, according to Equation 1 below.

$$Delta {E}_{\% T} = {[ {( \% T - \% {T}_{o} )}^{2} + {( {a}^{*} - {a}_{o}^{*} )}^{2} + {( {b}^{*} - {b}_{o}^{*} )}^{2} ]}^{0.5} \qquad Equation 1$$

The lower the Delta E_{% T} value, the less tint and color the sample possesses, in this case in the unactivated state. The activated % T at 35° C. indicates how dark a photochromic sample will get when exposed to actinic radiations, under warm, summer like conditions

The inventors surprisingly have discovered that indeno-fused naphthopyran compounds having a 7-position alkyl substitution can significantly decrease the Delta E_{% T} when combined with electron donating groups on 3,3′-position aryl groups as compared to structurally similar compounds with a 7-position alkoxy group. The indeno-fused compounds of the present invention also have been found to improve the activated darkness over structurally similar compounds having no substitution at the 7-position.

### SUMMARY

The present invention is directed to an indeno-fused naphthopyran having the following core skeletal structure (I):

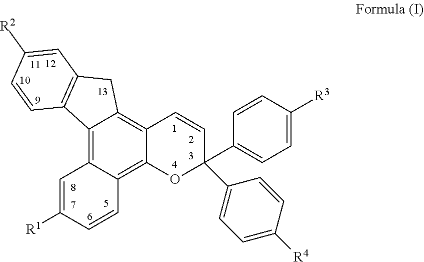

wherein
R¹ is a substituted or unsubstituted alkyl group;
R² is
   i. substituted or unsubstituted amino;
   ii. substituted or unsubstituted alkyl;
   iii. substituted or unsubstituted alkenyl;
   iv. substituted or unsubstituted alkynyl;
   v. substituted or unsubstituted aryl;
   vi. substituted or unsubstituted heteroaryl;
   vii. substituted or unsubstituted alkoxy;
   viii. substituted or unsubstituted aryloxy;
   ix. substituted or unsubstituted alkylthio;
   x. substituted or unsubstituted arylthio;
   xi. substituted or unsubstituted amide;
   xii. substituted or unsubstituted urea; or
   xiii. substituted or unsubstituted carbamate; and
   at least one of R³ and R⁴ is an electron donating group, wherein the sum of the Hammett σₚ values for R³ and R⁴ is less than −0.40, and
   wherein when R¹ is a substituted alkyl group, a carbon atom of the alkyl group is directly bonded to the 7-position carbon atom, and the carbon atom of the alkyl group bonded to the 7-position carbon atom is unsubstituted.

Compositions and articles comprising the indeno-fused naphthopyran are also provided.

The features that characterize the present invention are pointed out with particularity in the claims, which are annexed to and form a part of this disclosure. These and other features of the invention, its operating advantages, and the specific objects obtained by its use will be more fully understood from the following detailed description in which non-limiting embodiments of the invention are illustrated and described.

### BRIEF DESCRIPTION OF THE DRAWINGS

FIG. 1 illustrates a general scheme, Synthesis Part 1, of an exemplary method for synthesizing intermediates used to prepare photochromic compounds of the invention.

FIG. 2 illustrates a general scheme, Synthesis Part 2, of an exemplary method for preparing photochromic compounds of the invention.

### DETAILED DESCRIPTION

As used herein, the articles “a”, “an”, and “the” include plural referents unless otherwise expressly and unequivocally limited to one referent.

As used herein, the term “includes” is synonymous with “comprises.”

Unless otherwise indicated, all ranges or ratios disclosed herein are to be understood to encompass any and all subranges or sub-ratios subsumed therein. For example, a stated range or ratio of “1 to 10” should be considered to include any and all subranges between (and inclusive of) the minimum value of 1 and the maximum value of 10; that is, all subranges or subratios beginning with a minimum value of 1 or more and ending with a maximum value of 10 or less, such as but not limited to, 1 to 6.1, 3.5 to 7.8, and 5.5 to 10.

As used herein, unless otherwise indicated, left-to-right representations of linking groups, such as divalent linking groups, are inclusive of other appropriate orientations, such as, but not limited to, right-to-left orientations. For purposes of non-limiting illustration, the left-to-right representation of the divalent linking group

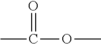

or equivalently —C(O)O—, is inclusive of the right-to-left representation thereof,

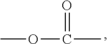

or equivalently —O(O)C— or —OC(O)—.

Other than in the operating examples, or where otherwise indicated, all numbers expressing quantities of ingredients, reaction conditions, and so forth used in the specification and claims are to be understood as modified in all instances by the term “about.” By “about” is meant plus or minus twenty-five percent of the stated value, such as plus or minus ten percent of the stated value. However, this should not be considered as limiting to any analysis of the values under the doctrine of equivalents.

As used herein, molecular weight values of polymers, such as weight average molecular weights (Mw) and number average molecular weights (Mn), are determined by gel permeation chromatography using appropriate standards, such as polystyrene standards.

As used herein, the term “polymer” means homopolymers (e.g., prepared from a single monomer species), copolymers (e.g., prepared from at least two monomer species), and graft polymers.

As used herein, the term “(meth)acrylate” and similar terms, such as “(meth)acrylic acid ester” means derivatives of acrylic acid and methacrylic acid, inclusive of acrylate esters, methacrylate esters, acrylamides, methacrylamides, acrylic acid, and methacrylic acid. As used herein, the term “(meth)acrylic acid” means methacrylic acid and/or acrylic acid.

The photochromic compounds of the present invention are, with some embodiments, also referred to herein as photochromic-dichroic compounds (such as, when they include one or more lengthening groups, such as L¹).

The photochromic compounds of the present invention, as described herein, including, but not limited to, photochromic compounds represented by Formula (I) and/or Formula (Ia), in each case can optionally further include one or more coproducts, resulting from the synthesis of such compounds.

As used herein, the term “photochromic” and similar terms, such as “photochromic compound” means having an absorption spectrum for at least visible radiation that varies in response to absorption of at least actinic radiation. Further, as used herein, the term “photochromic material” means any substance that is adapted to display photochromic properties (such as, adapted to have an absorption spectrum for at least visible radiation that varies in response to absorption of at least actinic radiation) and which includes at least one photochromic compound.

As used herein, the term “actinic radiation” means electromagnetic radiation that is capable of causing a response in a material, such as, but not limited to, transforming a photochromic material from one form or state to another, as will be discussed in further detail herein.

As used herein, the term “dichroic” means capable of absorbing one of two orthogonal plane polarized components of at least transmitted radiation more strongly than the other.

As used herein, the term “photochromic-dichroic” and similar terms, such as “photochromic-dichroic compound”, means possessing and/or providing both photochromic properties (i.e., having an absorption spectrum for at least visible radiation that varies in response to at least actinic radiation), and dichroic properties (i.e., capable of absorbing one of two orthogonal plane polarized components of at least transmitted radiation more strongly than the other).

As used herein, and unless stated otherwise or otherwise limited, the term “photochromic material” includes thermally reversible photochromic materials and compounds and non-thermally reversible photochromic materials and compounds. The term “thermally reversible photochromic compounds/materials,” as used herein, means compounds/materials capable of converting from a first state, for example a “clear state,” to a second state, for example a “colored state,” in response to actinic radiation, and reverting back to the first state in response to thermal energy. The term “non-thermally reversible photochromic compounds/materials,” as used herein, means compounds/materials capable of converting from a first state, for example a “clear state,” to a second state, for example a “colored state,” in response to actinic radiation, and reverting back to the first state in response to actinic radiation of substantially the same wavelength(s) as the absorption(s) of the colored state (e.g., discontinuing exposure to such actinic radiation).

As used herein, to modify the term “state,” the terms “first” and “second” are not intended to refer to any particular order or chronology, but instead refer to two different conditions or properties. For purposes of non-limiting illustration, the first state and the second state of a photochromic compound can differ with respect to at least one optical property, such as but not limited to the absorption of visible and/or UV radiation. Thus, according to various non-limiting embodiments disclosed herein, the photochromic compounds of the present invention can have a different absorption spectrum in each of the first state and second state. For example, while not limiting herein, a photochromic compound of the present invention can be clear in the first state and colored in the second state. Alternatively, a photochromic compound of the present invention can have a first color in the first state and a second color in the second state.

As used herein, the term “optical” means pertaining to or associated with light and/or vision. For example, according to various non-limiting embodiments disclosed herein, the optical article, element, or device can be chosen from ophthalmic articles, elements, and devices; display articles, elements, and devices; windows; mirrors; or active and passive liquid crystal cell articles, elements, and devices.

As used herein, the term “ophthalmic” means pertaining to or associated with the eye and vision. Non-limiting examples of ophthalmic articles or elements include corrective and non-corrective lenses, including single vision or multi-vision lenses, which can be either segmented or non-segmented multi-vision lenses (such as, but not limited to, bifocal lenses, trifocal lenses, and progressive lenses), as well as other elements used to correct, protect, or enhance (cosmetically or otherwise) vision, including without limitation, contact lenses, intra-ocular lenses, magnifying lenses, and protective lenses or visors.

As used herein, the term “display” means the visible or machine-readable representation of information in words, numbers, symbols, designs, or drawings. Non-limiting examples of display elements include screens, monitors, and security elements, such as security marks.

As used herein, the term “window” means an aperture adapted to permit the transmission of radiation there-through. Non-limiting examples of windows include automotive and aircraft transparencies, windshields, filters, shutters, and optical switches.

As used herein, the term “mirror” means a surface that specularly reflects a large fraction of incident light.

As used herein, the term “liquid crystal cell” refers to a structure containing a liquid crystal material that is capable of being ordered. A non-limiting example of a liquid crystal cell element is a liquid crystal display.

As used herein, spatial or directional terms, such as “left”, “right”, “inner”, “outer”, “above”, “below”, and the like, relate to the invention as it is depicted in the drawing figures. It is to be understood, however, that the invention can assume various alternative orientations and, accordingly, such terms are not to be considered as limiting.

As used herein, the terms “formed over”, “deposited over”, “provided over”, “applied over”, “residing over”, or “positioned over” mean formed, deposited, provided, applied, residing, or positioned on but not necessarily in direct (or abutting) contact with the underlying element, or surface of the underlying element. For example, a layer “positioned over” a substrate does not preclude the presence of one or more other layers, coatings, or films of the same or different composition located between the positioned or formed layer and the substrate.

As used herein, recitations relating to ring positions such as, but not limited to, position-x (e.g., position-3 or position-13) means a particular position in the ring structure, such as the core skeletal structure, of a chemical compound, such as the indeno-fused ring photochromic compounds of the present invention, and which are depicted herein in accordance with some embodiments by numbers within the ring structures of representative chemical formulas such as, but not limited to Formulas (I) and/or (Ia).

By “core skeletal structure” is meant a compound comprising at least the skeletal structure depicted in the associated Formula. The core skeletal structure is provided for purposes of identifying numbered ring positions. However, it is to be understood that, unless specifically shown to the contrary, the core skeletal structure(s) can have one or more atoms or one or more groups (not specifically illustrated on the corresponding Formula) bonded to one or more of the numbered ring positions on the core skeletal structure, which can be the same or different from one another.

The photochromic compounds of the present invention are referred to herein with reference to the term “core skeletal structure,” which can be represented by one or more formulas, such as but not limited to Formulas (I) and/or (Ia).

All documents or portions of documents, such as but not limited to issued patents and patent applications, referred to herein, and unless otherwise indicated, are to be considered to be “incorporated by reference” in their entirety.

As used herein, recitations of “substituted” group, means a group including, but not limited to, alkyl group, heterocycloalkyl group, aryl group, and/or heteroaryl group, in which at least one hydrogen thereof has been replaced or substituted with a group other than hydrogen, such as, but not limited to, halogen (e.g., F, Cl, I, and Br) group, cyano group, nitro group, alkyl group, alkenyl group, alkynyl group, haloalkyl group, perhaloalkyl group, heterocycloalkyl group, aryl group, heteroaryl group, alkoxy group, hydroxyl group, alkylthio group, arylthio group, phosphoric acid ester group, sulfonic acid group, sulfonic acid ester group, ketone group, aldehyde group, ester group, carboxylic acid group, carboxylate group, siloxane group, alkoxysilane group, polysiloxane group, amide group, amino group, carbamate group, carbonate group, urea group, polyester group, polyether group, polycarbonate group, polyurethane group, acrylate group, methacrylate group, aryl amino group, e.g., diphenyl amino; alkyl amino, e.g., dimethyl amino; cyclic amino, e.g., morpholino, piperidino, piperazino, or pyrrolidino; heteroaromatics, e.g., imidazole, pyrrole, indole, or carbazole; or combinations thereof; or any other group as long as it does not adversely impact upon the performance properties of the compound, e.g., the photochromic performance properties of the compound.

“Aryl group” refers to an aromatic cyclic monovalent hydrocarbon radical, and the term “aromatic” refers to a cyclically conjugated hydrocarbon with a stability (due to delocalization) that is significantly greater than that of a hypothetical localized structure. Examples of aryl groups include C₆-C₁₄ aryl groups, such as, but not limited to, phenyl, naphthyl, phenanthryl, and anthracenyl.

As used herein, recitations of “halo substituted” and related terms (such as, but not limited to, haloalkyl groups, haloalkenyl groups, haloalkynyl groups, haloaryl groups and halo-heteroaryl groups) means a group in which at least one, and up to and including all of the available hydrogen groups thereof is substituted with a halo group. The term “halo-substituted” is inclusive of “perhalo-substituted.” As used herein, the term perhalo-substituted group and related terms (such as, but not limited to, perhaloalkyl groups, perhaloalkenyl groups, perhaloalkynyl groups, perhaloaryl groups or perhalo-heteroaryl groups) means a group in which all of the available hydrogen groups thereof are substituted with a halo group. For example, perhalomethyl is —CX₃; perhalophenyl is —C₆X₅, where X represents one or more halo groups, such as, but not limited to F, Cl or Br.

As used herein, recitations of “linear or branched” groups, such as linear or branched alkyl, are herein understood to include: a methylene group or a methyl group; groups that are linear (or “straight chain”), such as linear C₁-C₂₅ alkyl groups; and groups that are appropriately branched, such as branched C₃-C₂₅ alkyl groups.

The term “alkyl,” as used herein, means linear or branched, cyclic or acyclic C₁-C₂₅ alkyl. Linear or branched alkyl can include C₁-C₂₅ alkyl, such as C₁-C₂₀ alkyl, such as C₂-C₁₀ alkyl, such as C₁-C₁₂ alkyl, such as C₁-C₆ alkyl. Examples of alkyl groups from which the various alkyl groups of the present invention can be selected from include, but are not limited to, those recited further herein. Alkyl groups can include “cycloalkyl” groups. The term “cycloalkyl,” as used herein, means groups that are appropriately cyclic, such as, but not limited to, C₃-C₁₂ cycloalkyl (including, but not limited to, cyclic C₅-C₇ alkyl, or cyclic C₃-C₁₀ alkyl) groups. Examples of cycloalkyl groups include, but are not limited to, those recited further herein. The term “cycloalkyl,” as used herein, also includes: bridged ring polycycloalkyl groups (or bridged ring polycyclic alkyl groups), such as, but not limited to, bicyclo[2.2.1]heptyl (or norbornyl) and bicyclo[2.2.2]octyl; and fused ring polycycloalkyl groups (or fused ring polycyclic alkyl groups), such as, but not limited to, octahydro-1H-indenyl, and decahydronaphthalenyl.

The term “heterocycloalkyl,” as used herein, means groups that are appropriately cyclic, such as, but not limited to, C₂-C₁₂ heterocycloalkyl groups, such as C₅-C₇ heterocycloalkyl groups, such as C₂-C₁₀ heterocycloalkyl groups, and which have at least one hetero atom in the cyclic ring, such as, but not limited to, O, S, N, P, and combinations thereof. Examples of heterocycloalkyl groups include, but are not limited to, imidazolyl, tetrahydrofuranyl, tetrahydropyranyl, and piperidinyl. The term “heterocycloalkyl,” as used herein, also includes: bridged ring polycyclic heterocycloalkyl groups, such as, but not limited to, 7-oxabicyclo[2.2.1]heptanyl; and fused ring polycyclic heterocycloalkyl groups, such as, but not limited to, octahydrocyclopenta[b]pyranyl, and octahydro-1H-isochromenyl.

The term “heteroaryl,” as used herein, includes, but is not limited to, C₃-C₁₈ heteroaryl, such as, but not limited to, C₃-C₁₀ heteroaryl (including fused ring polycyclic heteroaryl groups) and means an aryl group having at least one hetero atom in the aromatic ring, or in at least one aromatic ring in the case of a fused ring polycyclic heteroaryl group. Examples of heteroaryl groups include, but are not limited to, furanyl, pyranyl, pyridinyl, isoquinoline, and pyrimidinyl.

As used herein, the term “fused ring polycyclic-aryl-alkyl group” and similar terms such as, fused ring polycyclic-alkyl-aryl group, fused ring polycyclo-aryl-alkyl group, and fused ring polycyclo-alkyl-aryl group, means a fused ring polycyclic group that includes at least one aryl ring and at least one cycloalkyl ring that are fused together to form a fused ring structure. For purposes of non-limiting illustration, examples of fused ring polycyclic-aryl-alkyl groups include, but are not limited to indenyl, 9H-flourenyl, cyclopentanaphthenyl, and indacenyl.

The term “aralkyl,” as used herein, includes, but is not limited to, C₆-C₂₄ aralkyl, such as, but not limited to, C₆-C₁₀ aralkyl, and means an alkyl group substituted with an aryl group. Examples of aralkyl groups include, but are not limited to, benzyl and phenethyl.

Representative alkyl groups include, but are not limited to, methyl, ethyl, propyl, isopropyl, butyl, isobutyl, sec-butyl, tert-butyl, pentyl, neopentyl, hexyl, heptyl, octyl, nonyl, and decyl. Representative alkenyl groups include, but are not limited to, vinyl, allyl, and propenyl. Representative alkynyl groups include, but are not limited to, ethynyl, 1-propynyl, 2-propynyl, 1-butynyl, and 2-butynyl. Representative cycloalkyl groups include, but are not limited to, cyclopropyl, cyclobutyl, cyclopentyl, cyclohexyl, and cyclooctyl substituents. Representative heterocycloalkyl groups include, but are not limited to, imidazolyl, tetrahydrofuranyl, tetrahydropyranyl, and piperidinyl. Representative aryl groups include, but are not limited to, phenyl, naphthyl, anthracynyl, phenanthrenyl, and tetracenyl (including structural isomers thereof). Representative heteroaryl groups include, but are not limited to, furanyl, pyranyl, pyridinyl, isoquinolinyl, and pyrimidinyl. Representative aralkyl groups include, but are not limited to, benzyl and phenethyl.

The term “nitrogen-containing heterocycle,” as used herein, is a cyclic amino group which includes, but is not limited to, a nitrogen-containing ring wherein the nitrogen-containing ring is bonded through a ring nitrogen. Examples of nitrogen-containing heterocycles are those cyclic amino groups which include, but are not limited to, morpholino, piperidino, piperazino, and pyrrolidino; and heteroaromatics, such as imidazole, pyrrole, indole, and carbazole.

As used herein, “at least one of” is synonymous with “one or more of”, whether the elements are listed conjunctively or disjunctively. For example, the phrases “at least one of A, B, and C” and “at least one of A, B, or C” each mean any one of A, B, or C, or any combination of any two or more of A, B, or C. For example, A alone; or B alone; or C alone;
or A and B; or A and C; or B and C; or all of A, B, and C.

As used herein, “selected from” or “chosen from” are synonymous with “at least one of”, whether the elements are listed conjunctively or disjunctively. For example, the phrases “selected from A, B, and C” and “selected from A, B, or C” each mean any one of A, B, or C, or any combination of any two or more of A, B, or C. For example, A alone; or B alone; or C alone; or A and B; or A and C; or B and C; or all of A, B, and C.

The discussion of the invention may describe certain features as being “particularly” or “preferably” within certain limitations (e.g., “preferably”, “more preferably”, or “even more preferably”, within certain limitations). It is to be understood that the invention is not limited to these particular or preferred limitations but encompasses the entire scope of the disclosure.

The invention comprises, consists of, or consists essentially of, the following aspects of the invention, in any combination.

The indeno-fused naphthopyran compounds, according to the present invention, can be represented by one or more of the core skeletal structures described below. Each available numbered ring position (e.g., 5, 6, 8, 9, 10, 12, and/or 13) of the core skeletal structure of Formula (I) can have covalently bonded hydrogen thereto or a group other than hydrogen, for example, such as a group described herein. Examples of such groups are described below.

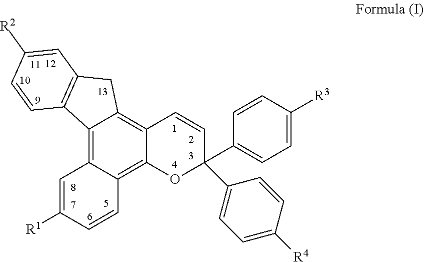

With reference to Formula (I), R¹ is a substituted or unsubstituted alkyl group, such as a C₁ to C₂₀ substituted or unsubstituted alkyl group or a substituted or unsubstituted C₁ to C₆ alkyl group. When R¹ is a substituted alkyl group, a carbon atom of the alkyl group is directly bonded to the 7-position carbon atom, and the carbon atom of the alkyl group, which is directly bonded to the 7-position carbon atom, is unsubstituted. R¹ also can be a substituted or unsubstituted alkyl group which optionally is interrupted with a heteroatom provided that the carbon atom at the 7-position is directly bonded to a carbon atom of the alkyl group. R¹ can include, but is not limited to, a substituted or unsubstituted alkyl group, such as methyl, ethyl, propyl, isopropyl, butyl, isobutyl, sec-butyl, tert-butyl, pentyl, neopentyl, or heptyl, each of which independently can be substituted or unsubstituted. Suitable substituent groups can include those described herein above. For example, each alkyl substituent can, in each case, be independently selected from one or more of halogen, cyano, nitro, alkenyl, alkynyl, heterocycloalkyl, aryl, heteroaryl, alkoxy, hydroxyl, alkylthio, arylthio, ketone, aldehyde, ester, carboxylic acid, carboxylate, siloxane, alkoxysilane, polysiloxane, amide, amino, carbamate, carbonate, urea, polyester group, polyether group, polycarbonate group, polyurethane group, an acrylate group, a methacrylate group, or combinations thereof, or any other group as long as it does not adversely impact upon the performance properties of the compound, e.g., the photochromic performance properties of the compound. Also, R₁ can be an unsubstituted C₁ to C₆ alkyl group.

Also, with reference to Formula (I), R² can be a substituted or unsubstituted amino, such as a primary amino group, a secondary amino group, for example, an alkyl amino or an aryl amino group, or a tertiary amino group, such as tertiary amino group having alkyl, and/or aryl substituents, or a cyclic amino group, such as a substituted or unsubstituted nitrogen-containing heterocycle; substituted or unsubstituted alkyl; substituted or unsubstituted alkenyl; substituted or unsubstituted alkynyl; substituted or unsubstituted aryl; substituted or unsubstituted heteroaryl; substituted or unsubstituted alkoxy; substituted or unsubstituted aryloxy; substituted or unsubstituted alkylthio; substituted or unsubstituted arylthio; substituted or unsubstituted amide including, for example, a substituted amide having aryl or alkyl substituents; substituted or unsubstituted urea; and substituted or unsubstituted carbamate. For purposes of the present invention, when R² is a substituted or unsubstituted amide group or a substituted or unsubstituted carbamate group, R² is bonded to the 11-position carbon atom through a nitrogen atom of the amide group or the carbamate group, as the case may be.

R² can be selected from the group consisting of substituted or unsubstituted aryl, substituted or unsubstituted amino, substituted or unsubstituted alkyoxy, substituted or unsubstituted aryloxy, substituted or unsubstituted alkylthio, and substituted or unsubstituted arylthio. Also, R² can be substituted or unsubstituted aryl.

Suitable substituent groups can include those described herein above. For example, each alkyl substituent, each heterocycloalkyl substituent, each aryl substituent, and each heteroaryl substituent herein can, in each case, be independently selected from one or more of halogen, cyano, nitro, alkyl, alkenyl, alkynyl, haloalkyl, perhaloalkyl, heterocycloalkyl, aryl, heteroaryl, alkoxy, hydroxyl, alkylthio, arylthio, phosphoric acid ester; sulfonic acid group; sulfonic acid ester, ketone, aldehyde, ester, carboxylic acid, carboxylate, siloxane, alkoxysilane, polysiloxane, amide, amine, carbamate, carbonate, urea, polyester group, polyether group, polycarbonate group, polyurethane group, an acrylate group, a methacrylate group, aryl amino, e.g., diphenyl amino; alkyl amino, e.g., dimethyl amino; cyclic amino, such as nitrogen-containing heterocycle compounds, e.g., morpholino, piperidino, piperazino, or pyrrolidino; heteroaromatics, e.g., imidazole, pyrrole, indole, or carbazole; or combinations thereof; or any other group as long as it does not adversely impact upon the performance properties of the compound, e.g., the photochromic performance properties of the compound.

With further reference to Formula (I), at least one of R³ and R⁴ is an electron donating group, wherein the sum of the Hammett σₚ values for R³ and R⁴ is less than −0.40. The relative strength of electron donor groups is frequently described by Hammett Sigma values, or σₚvalues. A list of Hammett σₚ values for various substituents can be found in C. Hansch, A. Leo, and R. W. Taft, “A Survey of Hammett Substituent Constants and Resonance and Field Parameters”, Chem. Rev., 1991, 91, 165-195, which disclosure is incorporated herein by reference. Hammett σₚ values for selected exemplary substituents are listed in Table 1 below.

TABLE 1
Hammett σₚ Values for Selected Substituents

| Substituent | σₚ value | Substituent | σₚ value |
|---|---|---|---|
| —H | 0 | -Phenyl | −0.01 |
| —CH₃ | −0.17 | —NHCOOEt | −0.15 |
| —OCH₃ | −0.27 | —F | 0.062 |
| —N(CH₃)₂ | −0.84 | —Cl | 0.23 |
| -Morpholino | −0.65 (est) | —SCH₃ | 0.0 |
| —OCOMe | 0.31 | —SPh | 0.07 |
| —COOC₂H₅ | 0.45 | —CN | 0.66 |
| —OH | −0.37 | —CF₃ | 0.54 |

For example, at least one of R³ and R⁴ can be substituted or unsubstituted amino. Also, at least one of R₃ and R₄ can be a cyclic amino group, such as a substituted or unsubstituted nitrogen-containing heterocycle, for example, a nitrogen-containing heterocycle selected from the group consisting of morpholino, piperidino, substituted piperazino, and pyrrolidino. Further, R³ and R⁴ can be the same or different, and each independently can be substituted or unsubstituted alkoxy, or substituted or unsubstituted amino. Additionally, at least one of R³ and R⁴ can be selected from the group consisting of methoxy, morpholino, piperazino, substituted piperazino, and dialkyl(C₁-C₆)amino.

Additionally or alternatively, the indeno-fused naphthopyran compounds of the present invention can be represented by the core skeletal structure of Formula (Ia):

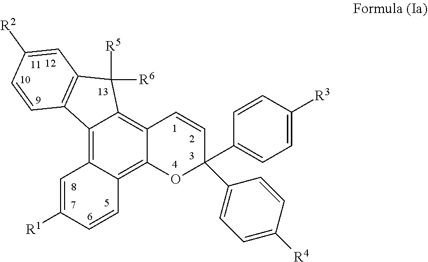

With reference to Formula (Ia), R¹, R², R³, and R⁴ are as previously described with respect to Formula (I).

As described above, the remaining numbered ring positions (e.g., 5, 6, 8, 9, 10, and/or 12) of the core skeletal structure of Formula (Ia) without a specifically shown substituent can have hydrogen covalently bonded thereto or a group other than hydrogen, for example, such as a group described herein.

With further reference to Formula (Ia), each of R⁵ and R⁶ is independently:
(i) hydrogen, substituted or unsubstituted alkyl, substituted or unsubstituted heterocycloalkyl, allyl, substituted or unsubstituted aryl, or substituted or unsubstituted heteroaryl;
(ii) alkoxy, hydroxyl, alkylthio, ketone, aldehyde, ester, carboxylic acid, carboxylate, siloxane, alkoxysilane, or polysiloxane;
(iii) a group comprising polyester, polyether, polycarbonate, polyurethane, or combinations thereof; or
(iv) R⁵ and R⁶ together form an aliphatic ring having 3 to 20 ring member carbon atoms, a condensed polycyclic ring having an aromatic ring or aromatic hetero ring condensed to the above aliphatic ring, a hetero ring having 3 to 20 ring member atoms, or a condensed polycyclic ring having an aromatic ring or aromatic hetero ring condensed to the above hetero ring, together with the 13-position carbon atom bonded thereto.

Each of R⁵ and R⁶ independently can be substituted or unsubstituted alkyl. For example, R⁵ and R⁶ independently can be selected from the group consisting of C₁ to C₆ linear or branched alkyl, and C₅ to C₁₀ cycloalkyl. Suitable substituent groups can include those substituent groups described herein above. For example, each alkyl substituent, each heterocycloalkyl substituent, each aryl substituent, and each heteroaryl substituent herein can, in each case, be independently selected from one or more of halogen, cyano, nitro, alkyl, alkenyl, alkynyl, haloalkyl, perhaloalkyl, heterocycloalkyl, aryl, heteroaryl, alkoxy, hydroxyl, alkylthio, arylthio, phosphoric acid ester, sulfonic acid group, sulfonic acid ester, ketone, aldehyde, ester, carboxylic acid, carboxylate, siloxane, alkoxysilane, polysiloxane, amide, amine, carbamate, carbonate, urea, polyester group, polyether group, polycarbonate group, polyurethane group, an acrylate group, a methacrylate group, aryl amino, e.g., diphenyl amino; alkyl amino, e.g., dimethyl amino; cyclic amino, e.g., morpholino, piperidino, or pyrrolidino; heteroaromatics, e.g., imidazole, pyrrole, indole, or carbazole; or combinations thereof; or any other group as long as it does not adversely impact upon the performance properties of the compound, e.g., the photochromic performance properties of the compound.

With reference to Formula (Ia), R¹ can be unsubstituted C₁ to C₆ alkyl; R² can be substituted or unsubstituted phenyl; R³ and R⁴ each independently can be C₁ to C₄ alkoxy or a nitrogen-containing heterocycle; and R⁵ and R⁶ each independently can be C₁ to C₄ alkyl.

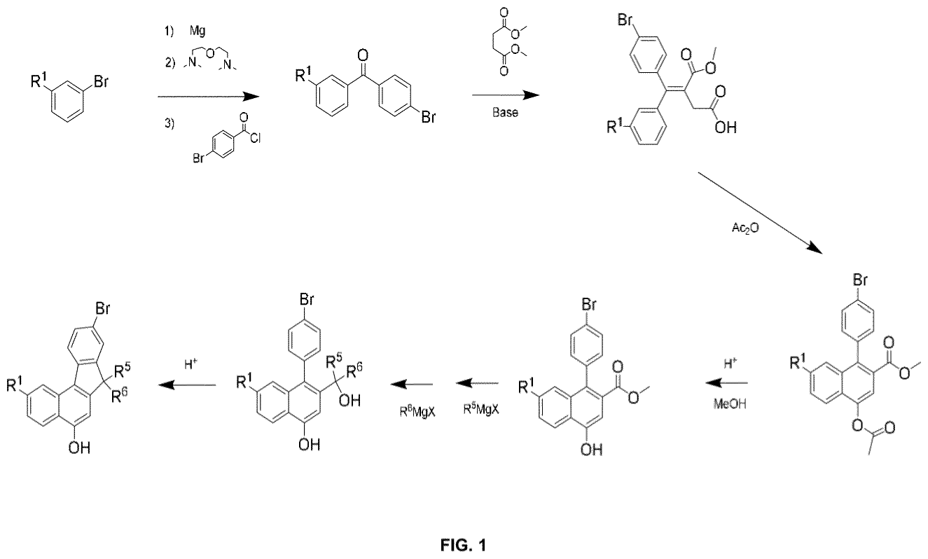

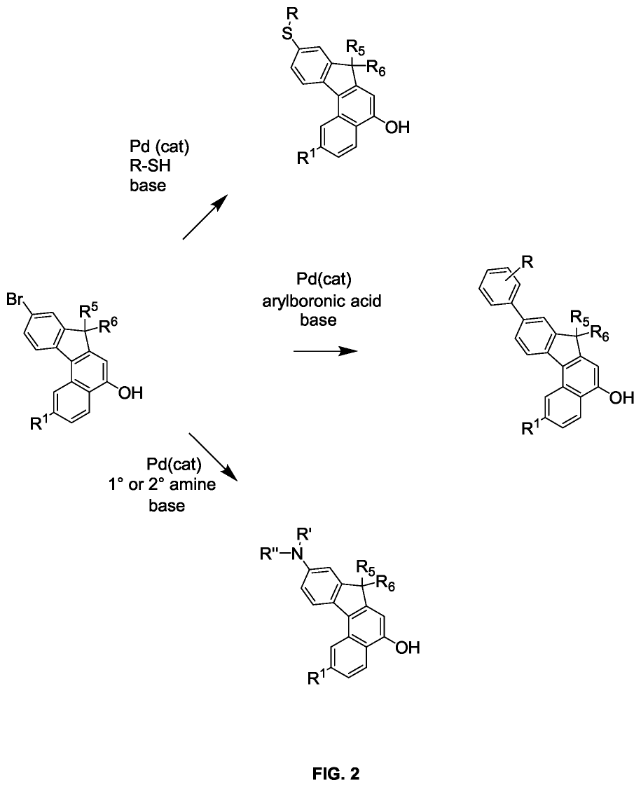

The indeno-fused naphthopyran compounds, according to the present invention, can be prepared in accordance with art-recognized methods. For purposes of non-limiting illustration and with reference to FIGS. 1 and 2, general synthetic schemes, Synthesis Part 1 and Synthesis Part 2, for the preparation of photochromic compounds, according to the present invention, is described as follows.

### Synthesis Part 1

### Step 1

A Grignard reagent can be prepared from an aryl bromide and reacted with an acid chloride to form the corresponding benzophenone.

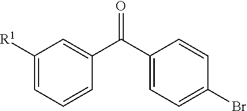

### Step 2

The benzophenone can be subject to Stobbe condensation to form the Stobbe acid.

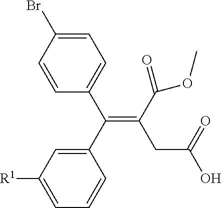

### Step 3

The carboxylic acid can undergo acid-catalyzed cyclization using acetic anhydride and a co-solvent at elevated temperatures.

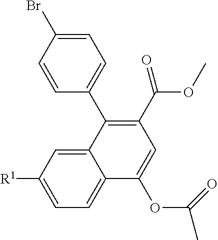

### Step 4

The acetoxy group can undergo acid-catalyzed methanolysis in methanol at elevated temperatures to give a naphthol.

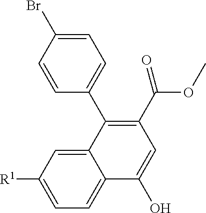

### Step 5

Reaction of methyl ester with two to six equivalents of Grignard reagent, optionally in the presence of a lanthanum(III)chloride-lithium chloride salt (LaCl₃-2LiCl), forms a tertiary alcohol.

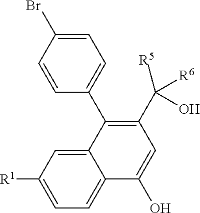

### Step 6

The tertiary alcohol can then undergo acid-catalyzed dehydration followed by intramolecular Friedel-Crafts reaction to give an indeno-fused naphthol. The naphthol serves as an intermediate to achieve various substitutions at the 11- and 3-positions of the target compounds, as shown in Synthesis Part 2 below.

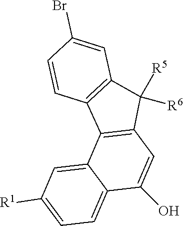

### Synthesis Part 2

The bromine can undergo cross-coupling with an aryl boronic acid using the standard Suzuki cross-coupling conditions. Alternatively, palladium-catalyzed cross-coupling of the bromine with an alkyl thiol or aryl thiol using Tris(dibenzylideneacetone)dipalladium(0) and Xantphos ligand at elevated temperatures can yield a thioether substitution. To introduce an amine at the position, palladium-catalyzed cross-coupling of the bromine with a primary or secondary amine using standard Buchwald-Hartwig cross-coupling conditions may be employed.

Each of the resulting naphthols can be reacted with a diaryl propargyl alcohol under known conditions to yield the desired indeno-fused naphthopyran.

FIG. 1 depicts the procedures for synthesizing indeno-fused naphthol intermediate compounds used to prepare the indeno-fused naphthopyran compounds of the present invention in accordance with the steps described above in Synthesis Part 1. FIG. 2 depicts the procedures for providing desired substituents at the 7- and 11-positions of the indeno-fused naphthopyran compounds of the present invention in accordance with the steps described above in Synthesis Part 2.

In accordance with the present invention, there is also provided a photochromic composition, which includes at least one indeno-fused naphthopyran compound according to the present invention, such as those represented by Formulas (I) and/or (Ia), as described previously herein.

The photochromic composition can include: (i) an organic material, in which the organic material is at least one of a polymeric material, an oligomeric material, or a monomeric material; and (ii) a photochromic indeno-fused naphthopyran compound according to the present invention, which is incorporated into at least a portion of the organic material. The photochromic compound can be incorporated into a portion of the organic material by methods including, but not limited to, at least one of blending or bonding the photochromic compound with the organic material or a precursor of the organic material. As used herein with reference to the incorporation of photochromic compounds into an organic material, the terms “blending” and “blended” mean that the photochromic compound/material is intermixed or intermingled with at least a portion of the organic material, but not bonded to the organic material. Further, as used herein with reference to the incorporation of photochromic compounds into an organic material, the terms “bonding” or “bonded” mean that the photochromic compound/material is linked, such as by one or more covalent bonds, to a portion of the organic material or a precursor thereof. For example, although not limiting herein, the photochromic material can be linked to the organic material through a reactive substituent.

When the organic material is a polymeric material, the photochromic indeno-fused naphthopyran compound can be incorporated into at least a portion of the polymeric material or at least a portion of the monomeric material or oligomeric material from which the polymeric material is formed. For example, photochromic indeno-fused naphthopyran compound(s) according to the present invention that have a reactive substituent can be bonded to an organic material such as a monomer, oligomer, or polymer having a group with which a reactive moiety may be reacted, or the reactive moiety can be reacted as a co-monomer in the polymerization reaction from which the organic material is formed, for example, in a co-polymerization process.

As discussed above, the photochromic compositions according to present invention can include an organic material chosen from a polymeric material, an oligomeric material, and/or a monomeric material, with some embodiments. Examples of polymeric materials that can be used with the photochromic compositions of the present invention include, but are not limited to: poly(carbonate), copolymers of ethylene and vinyl acetate; copolymers of ethylene and vinyl alcohol; copolymers of ethylene, vinyl acetate, and vinyl alcohol (such as those that result from the partial saponification of copolymers of ethylene and vinyl acetate); cellulose acetate butyrate; poly(urethane); poly(acrylate); poly(methacrylate); epoxies; aminoplast functional polymers; poly(anhydride); poly(urea urethane); N-alkoxymethyl(meth)acrylamide functional polymers; poly(siloxane); poly(silane); and combinations and mixtures thereof. Further classes and examples of polymeric materials that can be used with the photochromic compositions of the present invention include, but are not limited to, those disclosed at column 39, line 45 through column 40, line 67 of U.S. Pat. No. 9,028,728 B2, the recited portions of which being incorporated by reference herein.

Further, the indeno-fused naphthopyran photochromic compounds according to the present invention can be used in conjunction with one or more complementary conventional polymerizable or compatiblized photochromic compounds, such as, for example, those disclosed in U.S. Pat. No. 6,113,814 (at col. 2, line 39 to col. 8, line 41), and U.S. Pat. No. 6,555,028 (at col. 2, line 65 to col. 12, line 56), the recited portions of which being incorporated by reference herein. The indeno-fused naphthopyran photochromic compounds of the present invention can be used in combination with a mixture of other photochromic compounds. For example, although not limiting herein, mixtures of photochromic compounds can be used to attain certain activated colors, such as a near neutral gray or near neutral brown. See, for example, U.S. Pat. No. 5,645,767, col. 12, line 66 to col. 13, line 19, (the recited portions of which being incorporated by reference herein) which describes the parameters that define neutral gray and brown colors.

Examples of classes of other photochromic compounds that can be used in combination with the photochromic compounds of the present invention include, but are not limited to, indeno-fused naphthopyrans, naphtho[1,2-b]pyrans, naphtho[2,1-b]pyrans, spirofluoroeno[1,2-b]pyrans, phenanthrenopyrans, quinolinopyrans, fluoroanthenopyrans, spiropyrans, benzoxazines, naphthoxazines, spiro(indoline)naphthoxazines, spiro(indoline)pyridobenzoxazines, spiro(indoline)fluoranthenoxazines, spiro(indoline)quinoxazines, fulgides, fulgimides, diarylethenes, diarylalkylethenes, diarylalkenylethenes, thermally reversible photochromic compounds, and non-thermally reversible photochromic compounds, and mixtures thereof. Further examples of other photochromic compounds that can be used in combination with the photochromic compounds of the present invention include, but are not limited to, those disclosed at column 34, line 20 through column 35, line 13 of U.S. Pat. No. 9,028,728 B2, the recited portions of which being incorporated by reference herein.

The photochromic composition of the present invention can include at least one of a complementary photochromic material (including one or more of those other photochromic materials and compounds described previously herein), a photoinitiator, a thermal initiator, a polymerization inhibitor, a solvent, a light stabilizer, a heat stabilizer, a mold release agent, a rheology control agent, a leveling agent, a free radical scavenger, and/or an adhesion promoter.

The photochromic composition according to the present invention can be a photochromic coating composition. Photochromic coating compositions of the present invention can include: a photochromic indeno-fused naphthopyran compound according to the present invention, such as described previously herein with regard to Formulas (I) and/or (Ia); a resin composition that is optionally curable; and optionally a solvent. The photochromic coating composition can be in the form of art-recognized liquid coatings and powder coatings. The photochromic coating compositions of the present invention can be thermoplastic or thermosetting coating compositions. The photochromic coating composition can be a curable or thermosetting coating composition.

The curable resin composition of the curable photochromic coating compositions according to the present invention can include: a first reactant (or component) having functional groups, e.g., an epoxide functional polymer reactant; and a second reactant (or component) that is a crosslinking agent having functional groups that are reactive towards, and that can form covalent bonds with, the functional groups of the first reactant. The first and second reactants of the curable resin composition of the curable photochromic coating composition can each independently include one or more functional species, and are each present in amounts sufficient to provide cured photochromic coatings having a desirable combination of physical properties, e.g., smoothness, optical clarity, solvent resistance, and hardness.

Examples of curable resin compositions that can be used with the curable photochromic coating compositions according to the present invention include, but are not limited to: curable resin compositions including epoxide functional polymer (e.g., (meth)acrylic polymers containing residues of glycidyl (meth)acrylate) and epoxide reactive crosslinking agent (e.g., containing active hydrogens, such as hydroxyls, thiols, and amines); and curable resin compositions including active hydrogen functional polymer (e.g., hydroxy, thiol, and/or amine functional polymer) and capped (or blocked) isocyanate functional crosslinking agent. By “capped (or blocked) isocyanate functional crosslinking agent” is meant a crosslinking agent having two or more capped isocyanate groups that can decap (or deblock) under cure conditions (e.g., at elevated temperature) to form free isocyanate groups and free capping groups. The free isocyanate groups formed by decapping of the crosslinking agent are preferably capable of reacting and forming substantially permanent covalent bonds with the active hydrogen groups of the active hydrogen functional polymer (e.g., with the hydroxy groups of a hydroxy functional polymer). Further examples of curable resin compositions that can be used with the curable photochromic coating compositions according to the present invention include, but are not limited to, those disclosed in: paragraphs [0176] through [0190] of WO 2016/142496 A1; and paragraphs [0005], [0037] through [0051], [0056] through [0059], and [0063] through [0065] of WO 2017/030545 A1, the recited portions of which being incorporated by reference herein.

Curable photochromic coating compositions according to the present invention can, optionally, contain additives such as waxes for flow and wetting, flow control agents, e.g., poly(2-ethylhexyl)acrylate, adjuvant resin to modify and optimize coating properties, antioxidants and ultraviolet (UV) light absorbers. Examples of useful antioxidants and UV light absorbers include those available commercially from BASF under the trademarks IRGANOX and TINUVIN. These optional additives, when used, are typically present in amounts up to 20 percent by weight (e.g., from 0.5 to 10 percent by weight), based on total weight of resin solids of the curable resin composition.

Photochromic compositions, photochromic articles, and photochromic coating compositions according to the present invention can further include art-recognized additives that aid or assist in the processing and/or performance of the compositions or articles. Non-limiting examples of such additives include photoinitiators, thermal initiators, polymerization inhibitors, solvents, light stabilizers (such as, but not limited to, ultraviolet light absorbers and light stabilizers, such as hindered amine light stabilizers (HALS)), heat stabilizers, mold release agents, rheology control agents, leveling agents (such as, but not limited to, surfactants), free radical scavengers, adhesion promoters (such as hexanediol diacrylate and coupling agents), and combinations and mixtures thereof.

The photochromic indeno-fused naphthopyran compounds of the present invention can be used in amounts (or ratios) such that the compositions, organic material, or substrate (e.g., photochromic articles and photochromic coatings) into which the photochromic indeno-fused naphthopyran compounds are incorporated or otherwise connected exhibits desired optical properties. The amount and types of photochromic material can be selected such that the composition, organic material, or substrate is clear or colorless when the photochromic indeno-fused naphthopyran compound is in the closed-form (e.g., in the bleached or unactivated state), and can exhibit a desired resultant color when the photochromic compound (such as a photochromic indeno-fused naphthopyran of the present invention) is in the open-form (e.g., when activated by actinic radiation). The precise amount of the photochromic material that is utilized in the various photochromic compositions and articles described herein is not critical provided that a sufficient amount is used to produce the desired effect. The particular amount of the photochromic material used can depend on a variety of factors, such as but not limited to, the absorption characteristics of the photochromic compound, the color and intensity of the color desired upon activation, and the method used to incorporate or connect the photochromic material to the substrate.

Photochromic compositions according to the present invention can include the indeno-fused naphthopyran compound according to the present invention, including the compounds represented by Formulas (I) and/or (Ia) in an amount of from 0.01 to 40 weight percent, such as from 0.05 to 15 weight percent, such as from 0.1 to 5 weight percent, based on the weight of the photochromic composition. For purposes of further non-limiting illustration, the amount of the photochromic compound/material including the compounds represented by Formulas (I) and/or (Ia) that is incorporated into an organic material can range from 0.01 to 40 weight percent, such as from 0.05 to 15 weight percent, such as from 0.1 to 5 weight percent, based on the weight of the organic material.

The present invention also relates to photochromic articles that include one or more indeno-fused naphthopyran compounds according to the present invention, such as those represented by Formulas (I) and/or (Ia). The photochromic articles can be prepared by art-recognized methods, such as by imbibition methods, cast-in-place methods, coating methods, in-mold coating methods, over-mold methods, and lamination methods.

For example, the photochromic articles can be selected from ophthalmic articles, display articles, windows, mirrors, active liquid crystal cell articles, and passive liquid crystal cell articles.

Also, the photochromic articles of the present invention can be ophthalmic articles, and the ophthalmic articles can be selected from corrective lenses, non-corrective lenses, contact lenses, intra-ocular lenses, magnifying lenses, protective lenses, and visors.

Further, the photochromic articles of the present invention can be display articles, and the display articles can be selected from screens, monitors, and security elements.

The present invention is more particularly described in the following examples, which are intended as illustrative only, since numerous modifications and variations therein will be apparent to those skilled in the art.

### EXAMPLES

The following examples are provided to illustrate the preparation of the indeno-fused naphthopyran compounds of the present invention.

### Example 1

### Step 1

Magnesium turnings (5.33 g) and iodine (cat.) were vigorously stirred in anhydrous tetrahydrofuran (50 mL) under nitrogen for 30 minutes. The mixture was then cooled in a brine and ice bath to −5° C. 3-Bromotoluene (3.75 g) dissolved in anhydrous tetrahydrofuran (10 mL) was then added drop-wise into the reaction mixture. After 15 minutes, an exotherm was observed. Additional 3-bromotoluene (21.25 g) dissolved in anhydrous tetrahydrofuran (30 mL) was added drop-wise at a rate that maintained the temperature below 5° C. Upon completion, the mixture was held for one hour, then decanted into a second reaction vessel, leaving unreacted magnesium turnings behind. The second reaction vessel was stirred under nitrogen and placed in an ice bath. Bis[2-(N,N,-dimethylaminoethyl)]ether (23.42 g) dissolved in anhydrous tetrahydrofuran (20 mL) was added drop-wise over 10 minutes while maintaining the temperature below 5° C. Upon completion, the mixture was held for one hour. 4-Bromobenzoyl chloride (32.08 g) was then added to the reaction mixture in portions over 15 minutes while maintaining the temperature below 5° C. The reaction was held for two hours, then warmed to room temperature and held for 16 hours. The reaction mixture was poured into a mixture of 10% hydrochloric acid aqueous (v/v) and ice, and extracted twice with dichloromethane. The combined organic layers were washed with saturated aqueous sodium bicarbonate, dried over sodium sulfate, and concentrated under reduced pressure. The resulting residue was purified using flash chromatography on silica gel eluting with a mixture of 95% hexanes and 5% ethyl acetate. Fractions containing the desired product were combined and concentrated under reduced pressure. The resulting solid was triturated in a mixture of 93% hexanes and 7% ethyl acetate and then collected by filtration to give a white solid (4-bromophenyl)(m-tolyl)methanone (27.80 g, 69% yield).

### Step 2

The product from Step 1 (27.80 g) and dimethyl succinate (20.67 g) were combined and stirred vigorously in toluene (556 mL), and placed in an ice bath. Potassium tert-pentoxide solution (1.7 M in toluene, 101 mL) was added drop-wise over 30 minutes. After two additional hours, the reaction mixture was poured into ice water. Concentrated hydrochloric acid was added to the aqueous layer and ice until the pH reached 1. The aqueous layer was then extracted with dichloromethane twice. The combined organic layers were washed with brine, dried over sodium sulfate, and concentrated under reduced pressure to give an amber-colored oil (E)-4-(4-bromophenyl)-3-(methoxycarbonyl)-4-(m-tolyl)but-3-enoic acid (39.3 g, 100% yield).

### Step 3

The product from Step 2 (39.3 g) and acetic anhydride (41.2 g) was stirred in xylenes (118 mL). The solution was heated to 90° C. for 2 hours under a nitrogen gas atmosphere. The temperature was then increased to 140° C. for 1 hour. The solution was cooled to room temperature and concentrated under reduced pressure to give methyl 4-acetoxy-1-(4-bromophenyl)-7-methyl-2-naphthoate as an amber-colored oil (37.5 g, 90% yield).

### Step 4

The product from Step 3 (37.5 g) was dissolved in methanol (563 mL) and to it was added concentrated hydrochloric acid (3.75 mL). The solution was heated to 65° C. for 4 hours under nitrogen, then cooled to room temperature and slowly poured into a mixture of saturated aqueous sodium bicarbonate and ice while stirring vigorously. The mixture was extracted twice with dichloromethane. The combined organic layers were washed with brine, dried over sodium sulfate, and concentrated under reduced pressure. The resulting residue was chromatographed on silica gel eluting with eluting with methylene chloride. The more pure fractions, as determined by HPLC area %, were combined and concentrated under reduced pressure. The resulting oil was recrystallized in a mixture of 90% hexanes and 10% ethyl acetate to yield methyl 1-(4-bromophenyl)-4-hydroxy-7-methyl-2-naphthoate as a white crystalline solid (6.5 g, 19% yield).

### Step 5

Lanthanum chloride (7.93 g) and lithium chloride (4.11 g) were stirred in anhydrous tetrahydrofuran (90 mL) under nitrogen. The mixture was heated to 55° C. for 16 hours and then cooled to room temperature. The product from Step 4 (6.00 g) was added to the reaction mixture, then the vessel was cooled to −15° C. A solution of propylmagnesium chloride (2.0 M in diethyl ether, 49 mL) was added drop-wise into the reaction over 50 minutes while maintaining the temperature between −15 and −10° C. After holding 2 hours at this temperature, the reaction mixture was poured into a mixture of 10% hydrochloric acid aqueous (v/v) and ice, and extracted twice with ethyl acetate. The combined organic layers were washed with brine and dried over sodium sulfate, then passed over a short plug of silica gel while washing with ethyl acetate. The mother liquor was concentrated under reduced pressure to yield 4-(4-bromophenyl)-3-(4-hydroxyheptan-4-yl)-6-methylnaphthalen-1-ol as an amber-colored oil (6.0 g, 87% yield).

### Step 6

The product from Step 5 (6.91 g) and p-toluenesulfonic acid monohydrate (0.03 g) were dissolved in toluene (104 mL) and heated to 110° C. for 3 hours under nitrogen while employing a Dean-Stark trap. The solution was then cooled to 60° C. Bismuth triflate (0.11 g) was added and the solution heated to 90° C. for 45 minutes, followed by cooling to room temperature and filtering through a silica gel plug while washing with dichloromethane. The mother liquor was concentrated under reduced pressure, then recrystallized in hexanes to yield 9-bromo-2-methyl-7,7-dipropyl-7H-benzo[c]fluoren-5-ol as a white solid (5.3 g, 80% yield).

### Step 7

The product from Step 6 (1.67 g), 1-(3-fluoro-4-methoxyphenyl)-1-(4-morpholinophenyl)prop-2-yn-1-ol (2.79 g), and 4-dodecylbenzenesulfonic acid (0.13 g) were stirred in 1,2-dichloroethane (33 mL) and heated to 83° C. under a nitrogen gas atmosphere for 6 hours. It was then cooled to room temperature and filtered through a silica gel plug washing with dichloromethane. The mother liquor was concentrated under reduced pressure to yield 11-bromo-3-(3-fluoro-4-methoxyphenyl)-7-methyl-3-(4-morpholinophenyl)-13,13-dipropyl-3H,13H-indeno[2′,3′,3,4]naphtho[1,2-b]pyran as a dark-colored oil (2.80 g, 94% yield).

### Step 8

The product from Step 7 (0.90 g), phenylboronic acid (0.18 g), and potassium carbonate (0.37 g) were stirred in a mixture of N,N-dimethylacetamide (4 mL), deionized water (1.5 mL) and ethanol (1.5 mL). The mixture was sparged with nitrogen for 20 minutes and kept under nitrogen. Tetrakis(triphenylphosphine)palladium(0) (0.07 g) was added and the mixture was heated to 85° C. for 2 hours, then cooled to room temperature and poured into a mixture of 10% hydrochloric acid aqueous (v/v) and ice. It was then extracted with ethyl acetate twice. The combined organic layers were washed twice with brine, dried over sodium sulfate, and concentrated under reduced pressure. The resulting residue was purified using flash chromatography on silica gel eluting with a mixture of 75% hexanes and 25% ethyl acetate. Fractions containing the desired product were combined and concentrated under reduced pressure. The resulting residue was triturated in methanol and then collected by vacuum filtration to yield a light-blue solid (0.69 g, 77% yield). The ¹H NMR is consistent with the following structure.

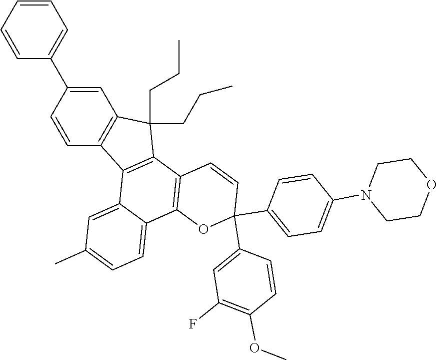 [SMILES: CCCC1(CCC)c2cc(-c3ccccc3)ccc2-c2c1c1c(c3ccc(C)cc23)OC(c2ccc(N3CCOCC3)cc2)(c2ccc(OC)c(F)c2)C=C1]

### Example 2

### Step 1

The product of Example 1, Step 6 (1.00 g) was subjected to the same conditions as Example 1, Steps 7 and 8, except 1-(3-fluoro-4-methoxyphenyl)-1-(4-morpholinophenyl)prop-2-yn-1-ol was replaced with an equimolar amount of 1-(4-methoxyphenyl)-1-(4-(4-((trifluoromethyl)sulfonyl)piperazin-1-yl)phenyl)prop-2-yn-1-ol in Step 7. A white solid was obtained (0.50 g, 45% yield). The ¹H NMR is consistent with the following structure.

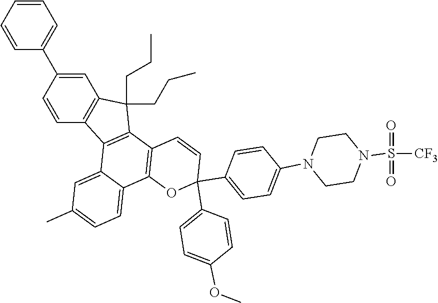 [SMILES: CCCC1(CCC)c2cc(-c3ccccc3)ccc2-c2c1c1c(c3ccc(C)cc23)OC(c2ccc(OC)cc2)(c2ccc(N3CCN(S(=O)(=O)C(F)(F)F)CC3)cc2)C=C1]

### Example 3

### Step 1

The product of Example 1, Step 6, was subject to the conditions of Example 1, Step 8 substituting an equimolar amount of (4-trifluoromethyl)phenylboronic acid in place of phenylboronic acid. Purification by flash chromatography on silica gel yielded a white solid 2-methyl-7,7-dipropyl-9-(4-(trifluoromethyl)phenyl)-7H-benzo[c]fluoren-5-ol (0.96 g, 83% yield).

### Step 2

The product from Step 1 (0.45 g), 1-(3-fluoro-4-methoxyphenyl)-1-(4-morpholinophenyl)prop-2-yn-1-ol (0.65 g) and 4-dodecylbenzenesulfonic acid (0.03 g) were stirred in 1,2-dichloroethane (9 mL) and heated to 83° C. under nitrogen for 6 hours, then cooled to room temperature followed by filtration through a silica gel plug with dichloromethane. The mother liquor was concentrated under reduced pressure, and purified using flash chromatography on silica gel eluting with a mixture of 80% hexanes and 20% ethyl acetate. The resulting residue was recrystallized in hexanes to yield a white solid (0.13 g, 17% yield). The ¹H NMR is consistent with the following structure.

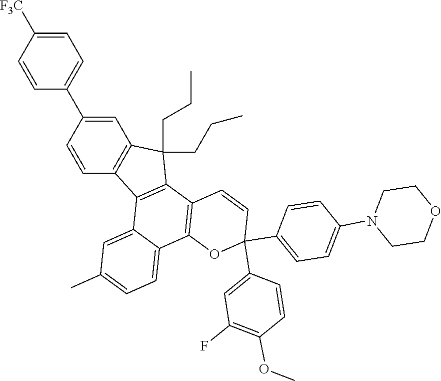 [SMILES: CCCC1(CCC)c2cc(-c3ccc(C(F)(F)F)cc3)ccc2-c2c1c1c(c3ccc(C)cc23)OC(c2ccc(N3CCOCC3)cc2)(c2ccc(OC)c(F)c2)C=C1]

### Example 4

### Step 1

The product of Example 1, Step 6 (3.26 g) was subjected to the same conditions as Example 1, Step 8, except phenylboronic acid was replaced with equimolar amount of 4-methoxyphenylboronic acid to yield a solid 9-(4-methoxyphenyl)-2-methyl-7,7-dipropyl-7H-benzo[c]fluoren-5-ol (2.44 g, 70% yield).

### Step 2

The product from Step 1 (5.00 g) was subject to the same conditions as Example 3, Step 2 substituting and equimolar amount of 1-(4-butoxyphenyl)-1-(4-morpholinophenyl)prop-2-yn-1-ol for 1-(3-fluoro-4-methoxyphenyl)-1-(4-morpholinophenyl)prop-2-yn-1-ol. The residue was recrystallized in a mixture of 35% ethyl acetate and 65% methanol to give a crystalline solid (5.93 g, 66% yield). The ¹H NMR is consistent with the following structure.

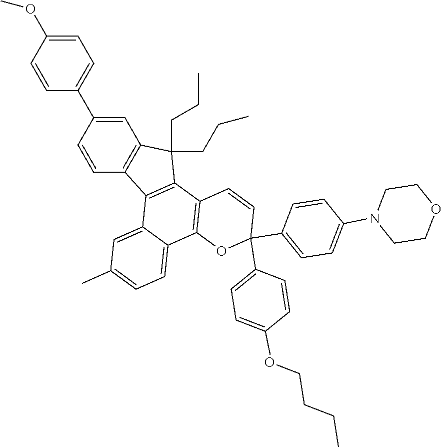 [SMILES: CCCCOc1ccc(C2(c3ccc(N4CCOCC4)cc3)C=Cc3c4c(c5cc(C)ccc5c3O2)-c2ccc(-c3ccc(OC)cc3)cc2C4(CCC)CCC)cc1]

### Example 5

### Step 1

The product of Example 1, Step 6 (3.00 g) was subjected to the same conditions as Example 1, Step 8, except phenylboronic acid was replaced with an equimolar amount of (2,4-dimethoxyphenyl)boronic acid to yield a solid 9-(2,4-dimethoxyphenyl)-2-methyl-7,7-dipropyl-7H-benzo[c]fluoren-5-ol (2.72 g, 80% yield).

### Step 2

The product from Step 1 (0.90 g) was subjected to the same conditions as Example 1, Step 7, except that 1-(3-fluoro-4-methoxyphenyl)-1-(4-morpholinophenyl)prop-2-yn-1-ol was replaced with an equimolar amount of 1-(4-methoxyphenyl)-1-(4-morpholinophenyl)prop-2-yn-1-ol to yield a white solid (0.76 g, 51% yield). The ¹H NMR is consistent with the following structure.

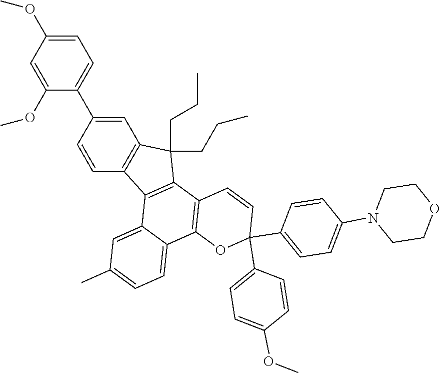 [SMILES: CCCC1(CCC)c2cc(-c3ccc(OC)cc3OC)ccc2-c2c1c1c(c3ccc(C)cc23)OC(c2ccc(OC)cc2)(c2ccc(N3CCOCC3)cc2)C=C1]

### Example 6

### Step 1

The product of Example 1, Step 6 was subjected to the same conditions as Example 1, Step 8 to yield a white solid with a ¹H NMR consistent with 2-methyl-9-phenyl-7,7-dipropyl-7H-benzo[c]fluoren-5-ol.

### Step 2

The product from Step 1 (0.40 g) was subjected to the same conditions as Example 3, Step 2, except that 1-(3-fluoro-4-methoxyphenyl)-1-(4-morpholinophenyl)prop-2-yn-1-ol was replaced with an equimolar amount of 1-(4-methoxyphenyl)-1-(4-morpholinophenyl)prop-2-yn-1-ol to yield a white solid (0.37 g, 53% yield). The ¹H NMR is consistent with the following structure.

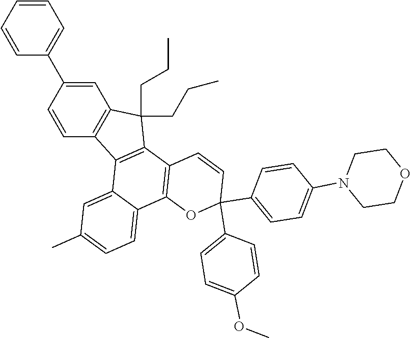 [SMILES: CCCC1(CCC)c2cc(-c3ccccc3)ccc2-c2c1c1c(c3ccc(C)cc23)OC(c2ccc(OC)cc2)(c2ccc(N3CCOCC3)cc2)C=C1]

### Example 7

### Step 1

The product of Example 1, Step 6 (5.00 g) and N,N-diisopropylethylamine (4.74 g) were stirred in toluene (30 mL). The mixture was sparged with nitrogen for 20 minutes and then placed under a blanket of nitrogen. Tris(dibenzylideneacetone)dipalladium(0) (0.78 g) and 4,5-bis(diphenylphosphino)-9,9-dimethylxanthene (1.06 g) were added to the reaction mixture. Next, 2,6-Dimethylbenzenethiol (3.38 g) was added to the mixture drop-wise. The reaction mixture was rapidly heated to 110° C. and held at that temperature for 17 hours. It was then cooled to room temperature and filtered over a plug of silica gel washing with a mixture of 75% hexanes and 25% ethyl acetate. The mother liquor was concentrated under reduced pressure. The resulting residue was purified using flash chromatography on silica gel eluting with a mixture of 80% hexanes and 20% ethyl acetate. Fractions containing the desired product were combined and concentrated under reduced pressure to give an oil 9-((2,6-dimethylphenyl)thio)-2-methyl-7,7-dipropyl-7H-benzo[c]fluoren-5-ol.

### Step 2

The product of Step 1 was subjected to the same conditions as Example 5, Step 2, and precipitated from methanol to yield a solid. The ¹H NMR is consistent with the following structure.

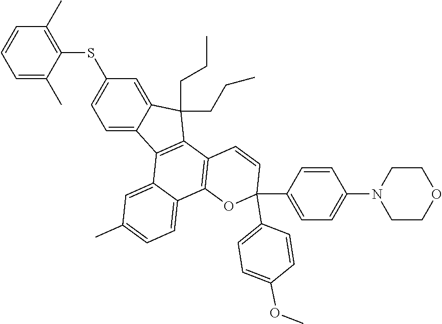 [SMILES: CCCC1(CCC)c2cc(Sc3c(C)cccc3C)ccc2-c2c1c1c(c3ccc(C)cc23)OC(c2ccc(OC)cc2)(c2ccc(N3CCOCC3)cc2)C=C1]

### Step 1

The product from Example 1, Step 6 (5.00 g), morpholine (2.13 g), and potassium tert-butoxide (2.74 g) were stirred in 1,2-dimethoxyethane (40 mL), sparged with nitrogen for 20 minutes, and held under a nitrogen atmosphere. Bis(tri-tert-butylphosphine)palladium(0) (0.44 g) was then added and the mixture heated to 85° C. for 16 hours. The mixture was then cooled to room temperature and water (100 mL), citric acid (15 g), and toluene (100 mL) were added. The resulting organic layer was filtered through a plug of magnesium sulfate and concentrated under reduced pressure. The resulting residue was purified by flash chromatography on silica gel, eluting with a mixture of hexanes 70% and ethyl acetate 30%. Fractions containing desired product were combined and concentrated under reduced pressure to give a white solid 2-methyl-9-morpholino-7,7-dipropyl-7H-benzo[c]fluoren-5-ol.

### Step 2

The product from Step 1 (3.56 g) was subjected to the same conditions as Example 5, Step 2 to give a solid. The ¹H NMR is consistent with the following structure.

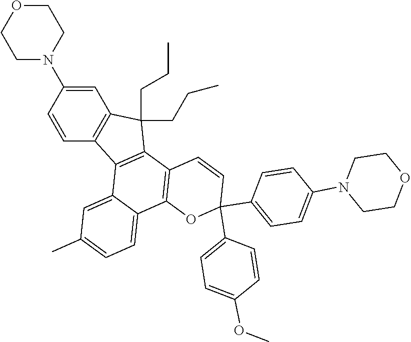 [SMILES: CCCC1(CCC)c2cc(N3CCOCC3)ccc2-c2c1c1c(c3ccc(C)cc23)OC(c2ccc(OC)cc2)(c2ccc(N3CCOCC3)cc2)C=C1]

### Example 9

### Step 1

2-bromo-9-methoxy-7,7-dipropyl-7H-benzo[c]fluoren-5-ol](5.0 g) was stirred in toluene (20 mL/g). The mixture was sparged with nitrogen for 20 minutes and kept under nitrogen. 1,1-Bis(diphenylphosphino)ferrocene dichloropalladium(II) (0.20 g) and tris(dibenzylideneacetone)dipalladium(0) (0.11 g) were added to the mixture. A 1.4 M solution of methylmagnesium bromide in THF/toluene (26 mL) was added drop-wise over 10 minutes. The reaction mixture was subsequently heated to 90° C. for 15 minutes, cooled to room temperature, then into ice water and acidified with dilute hydrochloric acid until the pH was 5. The reactions mixture was then extracted with ethyl acetate. The organic layer was concentrated under reduced pressure to dryness. The resulting residue was dissolved in a minimal amount of dichloromethane and then filtered through a plug of silica gel, washing with dichloromethane. The mother liquor was concentrated under reduce pressure to a white solid 9-methoxy-2-methyl-7,7-dipropyl-7H-benzo[c]fluoren-5-ol (4.1 g, 97% yield).

### Step 2

The product from Step 1 was subjected to the same conditions as Example 5, Step 2 to yield a solid. The ¹H NMR consistent with the following structure.

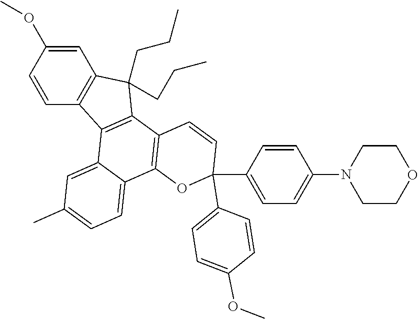 [SMILES: CCCC1(CCC)c2cc(OC)ccc2-c2c1c1c(c3ccc(C)cc23)OC(c2ccc(OC)cc2)(c2ccc(N3CCOCC3)cc2)C=C1]

### Example 10

### Step 1

The product of Example 1, Step 6 (2.00 g) was subjected to the same conditions as Example 1, Step 7, except 1-(3-fluoro-4-methoxyphenyl)-1-(4-morpholinophenyl)prop-2-yn-1-ol was replaced with an equimolar amount of 1-(4-butoxyphenyl)-1-(4-morpholinophenyl)prop-2-yn-1-ol to give a dark-colored oil 11-bromo-3-(4-butoxyphenyl)-7-methyl-3-(4-morpholinophenyl)-13,13-dipropyl-3H,13H-indeno[2′,3′,3,4]naphtho[1,2-b]pyran (3.64 g, 98% yield).

### Step 2

The product of Step 1 (3.64 g), 2-pyrrolidinone (2.05 g), and potassium phosphate, tribasic (3.06 g) were stirred in 1,4-dioxane (55 mL). The mixture was sparged with nitrogen gas for 20 minutes and subsequently placed under a nitrogen gas atmosphere. 2-di-tert-butylphosphino-2′,4′,6′-triisopropylbiphenyl (0.51 g) and tris(dibenzylideneacetone)dipalladium(0) (0.44 g) were added and then the mixture was heated to 90° C. for 2 hours. The mixture was then cooled to room temperature and poured into a mixture of dilute aqueous hydrochloric acid and ice. It was then extracted with ethyl acetate twice. The combined organic layers were then washed with a solution of saturated aqueous sodium bicarbonate, dried over sodium sulfate, and concentrated under reduced pressure to dryness. The resulting residue was purified by flash chromatography on silica gel, eluting with a mixture of 35% ethyl acetate and 65% hexanes. Fractions containing desired product were combined and concentrated under reduced pressure to dryness. The resulting residue was recrystallized in a mixture of 25% ethyl acetate and 75% methanol. The light-blue crystalline solid was isolated by filtration under vacuum to give (2.00 g, 55% yield). The ¹H NMR is consistent with the following structure.

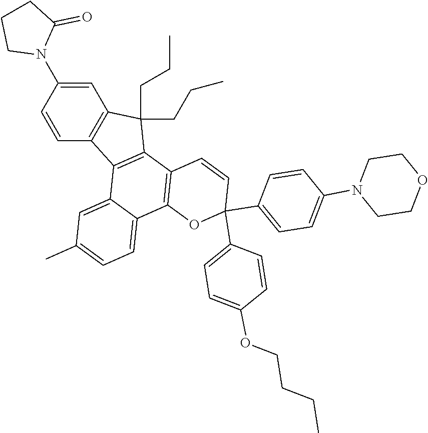 [SMILES: CCCCOc1ccc(C2(c3ccc(N4CCOCC4)cc3)C=Cc3c4c(c5cc(C)ccc5c3O2)-c2ccc(N3CCCC3=O)cc2C4(CCC)CCC)cc1]

### Example 11

### Step 1

The same conditions were employed as in Example 1, Step 1, except that 3-bromotoluene was replaced with 1-bromo-3-(tert-buty)benzene to give a white solid (4-bromophenyl)(3-(tert-butyl)phenyl)methanone (18.99 g, 74% yield).

### Step 2

The product from Step 1 (18.90 g) was treated to conditions of Example 1, Step 2 to yield an amber-colored oil (E)-4-(4-bromophenyl)-4-(3-(tert-butyl)phenyl)-3-(methoxycarbonyl)but-3-enoic acid (22.1 g, 86% yield).

### Step 3

The product from Step 2 (22.05 g) was treated to conditions of Example 1, Step 3 to yield an amber-colored oil methyl-4-acetoxy-1-(4-bromophenyl)-7-(tert-butyl)-2-naphthoate (18.25 g, 78% yield).

### Step 4

The product from Step 3 (37.5 g) was treated to conditions of Example 1, Step 4 to give a white solid methyl 1-(4-bromophenyl)-7-(tert-butyl)-4-hydroxy-2-naphthoate (3.4 g, 21% yield).

### Step 5

The product from Step 4 (3.31 g) was treated to the same conditions as Example 1,

Step 5 to yield an amber-colored oil 4-(4-bromophenyl)-6-(tert-butyl)-3-(4-hydroxyheptan-4-yl)naphthalen-1-ol (3.62 g, 96% yield).

### Step 6

The product from Step 5 (3.60 g) was treated to the conditions of Example 1, Step 6 to give a white solid 9-bromo-2-(tert-butyl)-7,7-dipropyl-7H-benzo[c]fluoren-5-ol (3.00 g, 87% yield).

### Step 7

The product from Step 6 (0.80 g) was subjected to the same conditions as Example 3, Step 1, except (4-trifluoromethyl)phenylboronic acid was replaced by equimolar amounts of (2,4-dimethoxyphenyl)boronic acid to yield a white solid 2-(tert-butyl)-9-(2,4-dimethoxyphenyl)-7,7-dipropyl-7H-benzo[c]fluoren-5-ol (0.81 g, 90% yield).

### Step 8

The product from Step 7 (0.81 g), was subjected to the same conditions as Example 4, Step 2, except that 1-(4-butoxyphenyl)-1-(4-morpholinophenyl)prop-2-yn-1-ol was replaced with equimolar amounts of 1-(4-methoxyphenyl)-1-(4-morpholinophenyl)prop-2-yn-1-ol to yield a solid (55 g, 42% yield). The ¹H NMR is consistent with the following structure.

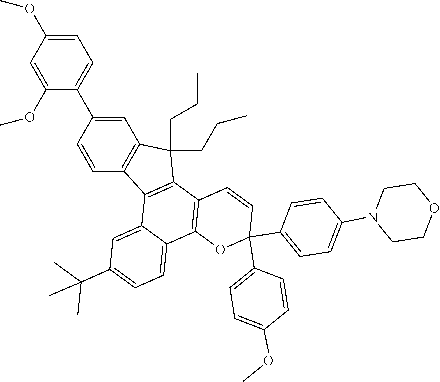 [SMILES: CCCC1(CCC)c2cc(-c3ccc(OC)cc3OC)ccc2-c2c1c1c(c3ccc(C(C)(C)C)cc23)OC(c2ccc(OC)cc2)(c2ccc(N3CCOCC3)cc2)C=C1]

### Part 2: Results

Each of the photochromic dyes from Examples 1 through 11 and each comparative example CE1 to CE10 were incorporated into a polyurethane coating system as described in U.S. Pat. No. 8,608,988 examples 1-3 at the same mol % and applied at the same coating thickness to 2″×2″ test chips made from CR-39® monomer (PPG Industries, Inc.). All coated test chips were cured at 125° C. for 1 hour.

Each of the coated test chips (hereinafter “test samples”) was conditioned by first being exposed to 365-nanometer ultraviolet light for 10 minutes at a distance of about 14 centimeters to activate the photochromic materials within the coating. The UVA (315 to 380 nm) irradiance at the test sample was measured with a LICOR® Model Li-1800 spectroradiometer and found to be 22.2 watts per square meter. Each of the test samples was then placed under a 500 watt, high intensity halogen lamp for 10 minutes at a distance of about 36 centimeters to bleach (inactivate) the photochromic materials. The illuminance at the test samples was measured with the LICOR® spectroradiometer and found to be 21.9 Klux. The test samples then were kept in a dark environment at room temperature (i.e., from 70 to 75° F., or 21 to 24° C.) for at least 1 hour prior to testing on an optical bench. Prior to optical bench measurement, the test samples were measured for ultraviolet absorbance at 390 nanometers.

Percent transmission for Examples 1 through 11, and for each comparative example CE1 through CE10 was determined using the CIE Y value in accordance with CIE 15: 2004 colorimetry using a D 65 illuminant and 100 observer. The a* and b* values, as used herein in the specification and the claims, refers to the a* and b* values measured in accordance with in accordance with CIE 15: 2004 space colorimetry, employing a D 65 illuminant and 100 observer, using the Hunter UltraScan Pro unit. The % T, a*, and b* values are for the photochromic dye containing samples and the % Tₒ, a*₀, and b*ₒ values are from a sample that was prepared with no photochromic dyes in the polyurethane coating. Delta E_{% T} was calculated according to the equation below.

$$Delta {E}_{\% T} = {[ {( \% T - \% {T}_{o} )}^{2} + {( {a}^{*} - {a}_{0}^{*} )}^{2} + {( {b}^{*} - {b}_{0}^{*} )}^{2} ]}^{0.5}$$

The BMP optical bench was fitted with two 150-watt ORIEL Model #66057 Xenon arc lamps at right angles to each other. The light path from Lamp 1 was directed through a 3 mm SCFIOTT® KG-2 band-pass filter and appropriate neutral density filters that contributed to the required UV and partial visible light irradiance level. The light path from Lamp 2 was directed through a 3 mm SCFIOTT® KG-2 band-pass filter, a SCFIOTT® short band 400 nm cutoff filter, and appropriate neutral density filters in order to provide supplemental visible light illuminance. A 2 inch×2 inch 50% polka dot beam splitter, at 450 to each lamp, is used to mix the two beams. The combination of neutral density filters and voltage control of the Xenon arc lamp were used to adjust the intensity of the irradiance. Proprietary software, i.e., BMPSoft version 2.1e, was used on the BMP to control timing, irradiance, air cell and sample temperature, shuttering, filter selection, and response measurement. A ZEISS® spectrophotometer, Model MCS 501, with fiber optic cables for light delivery through the test sample was used for response and color measurement. Photopic response measurements were collected on each test samples. The power output of the optical bench, i.e., the dosage of light that the test sample was exposed to, was adjusted to 6.7 Watts per square meter (W/m²) UVA, integrated from 315-380 nm and 50 Klux illuminance, integrated from 380-780 nm. Measurement of this power setpoint was made using an irradiance probe and the calibrated Zeiss spectrophotometer. The sample cell was fitted with a quartz window and self-centering sample holder. The temperature in the sample cell was controlled at 23° C. through the software with a modified Facis, Model FX-10, environment simulator. Measurement of the test sample's dynamic photochromic response and color measurements was made using the same Zeiss spectrophotometer, with fiber optic cables for light delivery from a tungsten halogen lamp and through the test sample. The collimated monitoring light beam from the fiber optic cable was maintained perpendicular to the test sample while passing through the sample and directed into a receiving fiber optic cable assembly attached to the spectrophotometer. The exact point of placement of the sample in the sample cell was where the activating xenon arc beam and the monitoring light beam intersected to form two concentric circles of light. The angle of incidence of the xenon arc beam at the sample placement point was =30° from perpendicular.

Response measurements, in terms of a change in optical density (AOD) from the unactivated or bleached state to the activated or colored state were determined by establishing the initial unactivated transmittance, opening the shutter from the Xenon lamp(s) and measuring the transmittance through activation at selected intervals of time. The AOD at saturation is after 15 minutes of activation and the Fade Half Life (“T½”) value is the time interval in seconds for the AOD of the activated form of the photochromic material in the coating to reach one half the fifteen minute AOD at 73.4° F. (23° C.), after removal of the activating light source.

TABLE A

| | | | | | | | | | | Fade |
|---|---|---|---|---|---|---|---|---|---|---|
| | Example | Bleach | | | | 23° C. Activated | | | 35° C. | T1/2 |
| | # | Y | a* | b* | ΔE_{%T} | Y | a* | b* | Y | (Sec) |
| 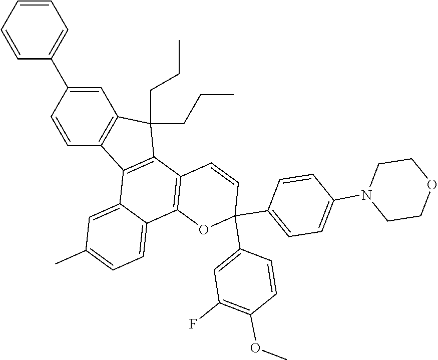 [SMILES: CCCC1(CCC)c2cc(-c3ccccc3)ccc2-c2c1c1c(c3ccc(C)cc23)OC(c2ccc(N3CCOCC3)cc2)(c2ccc(OC)c(F)c2)C=C1] | 1 | 89.3 | −1.7 | 2.3 | 4.0 | 11.0 | −0.2 | −28.5 | 23.5 | 42 |
| 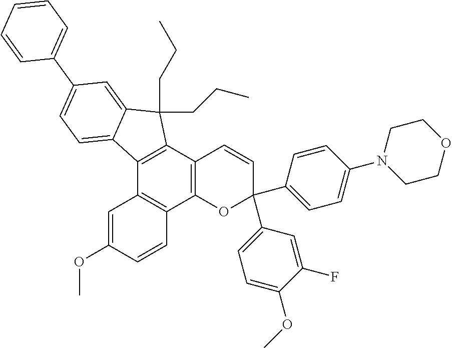 [SMILES: CCCC1(CCC)c2cc(-c3ccccc3)ccc2-c2c1c1c(c3ccc(OC)cc23)OC(c2ccc(N3CCOCC3)cc2)(c2ccc(OC)c(F)c2)C=C1] | CE1 | 86.2 | −1.4 | 2.2 | 6.5 | 7.4 | 4.9 | −19.3 | 18.5 | 56 |
| 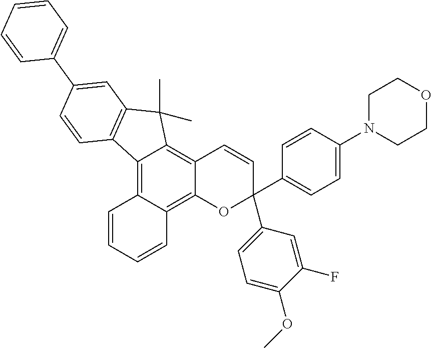 [SMILES: COc1ccc(C2(c3ccc(N4CCOCC4)cc3)C=Cc3c4c(c5ccccc5c3O2)-c2ccc(-c3ccccc3)cc2C4(C)C)cc1F] | CE1A | 90.6 | −1.8 | 3.0 | 3.6 | 13.9 | −2.1 | −32.2 | 29.0 | 38 |
| 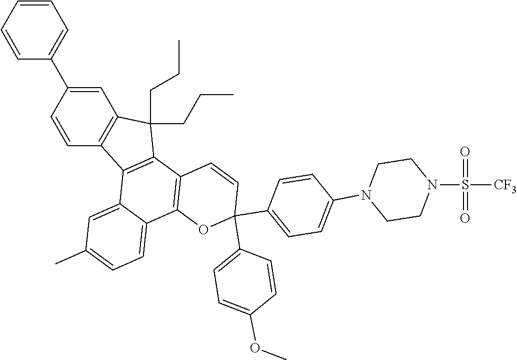 [SMILES: CCCC1(CCC)c2cc(-c3ccccc3)ccc2-c2c1c1c(c3ccc(C)cc23)OC(c2ccc(OC)cc2)(c2ccc(N3CCN(S(=O)(=O)C(F)(F)F)CC3)cc2)C=C1] | 2 | 88.9 | −1.7 | 2.5 | 4.5 | 12.0 | −2.2 | −22.5 | 24.8 | 39 |
| 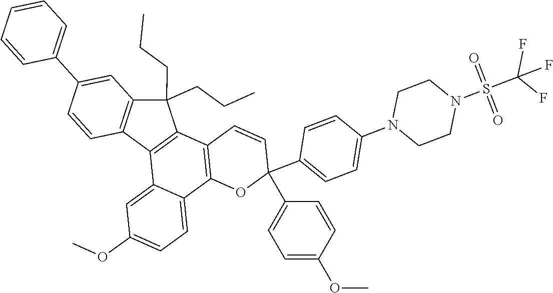 [SMILES: CCCC1(CCC)c2cc(-c3ccccc3)ccc2-c2c1c1c(c3ccc(OC)cc23)OC(c2ccc(OC)cc2)(c2ccc(N3CCN(S(=O)(=O)C(F)(F)F)CC3)cc2)C=C1] | CE2 | 85.5 | −1.4 | 2.3 | 7.2 | 8.4 | 3.8 | −12.5 | 19.6 | 57 |
| 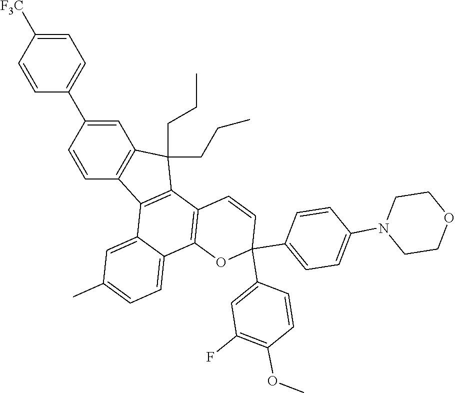 [SMILES: CCCC1(CCC)c2cc(-c3ccc(C(F)(F)F)cc3)ccc2-c2c1c1c(c3ccc(C)cc23)OC(c2ccc(N3CCOCC3)cc2)(c2ccc(OC)c(F)c2)C=C1] | 3 | 89.2 | −1.9 | 2.8 | 4.5 | 14.6 | 1.2 | −28.4 | 28.4 | 34 |
| 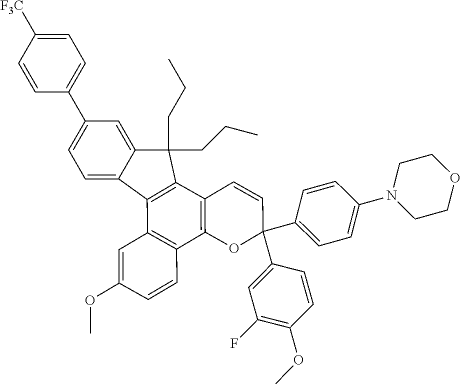 [SMILES: CCCC1(CCC)c2cc(-c3ccc(C(F)(F)F)cc3)ccc2-c2c1c1c(c3ccc(OC)cc23)OC(c2ccc(N3CCOCC3)cc2)(c2ccc(OC)c(F)c2)C=C1] | CE3 | 86.7 | −2.0 | 2.9 | 6.5 | 9.8 | 6.2 | −21.3 | 21.9 | 45 |
| 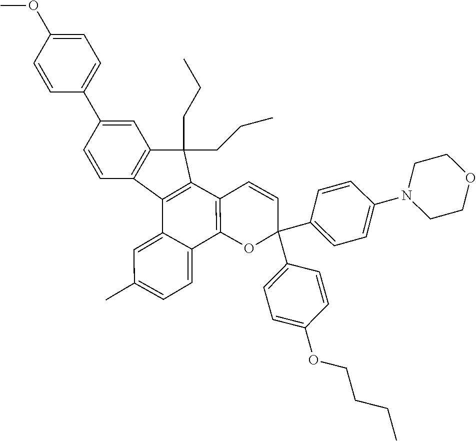 [SMILES: CCCCOc1ccc(C2(c3ccc(N4CCOCC4)cc3)C=Cc3c4c(c5cc(C)ccc5c3O2)-c2ccc(-c3ccc(OC)cc3)cc2C4(CCC)CCC)cc1] | 4 | 87.0 | −2.8 | 2.8 | 6.5 | 13.3 | −11.4 | −23.6 | 26.0 | 38 |
| 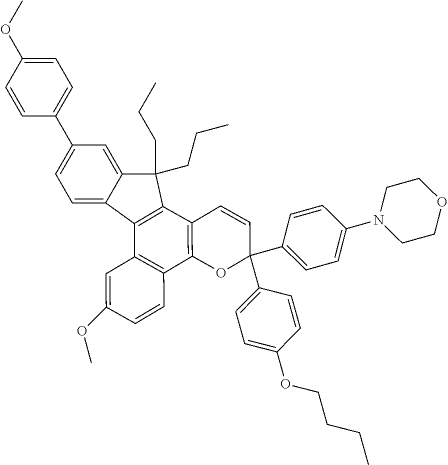 [SMILES: CCCCOc1ccc(C2(c3ccc(N4CCOCC4)cc3)C=Cc3c4c(c5cc(OC)ccc5c3O2)-c2ccc(-c3ccc(OC)cc3)cc2C4(CCC)CCC)cc1] | CE4 | 84.3 | −2.1 | 1.8 | 8.4 | 9.3 | −5.9 | −17.8 | 20.4 | 52 |
| 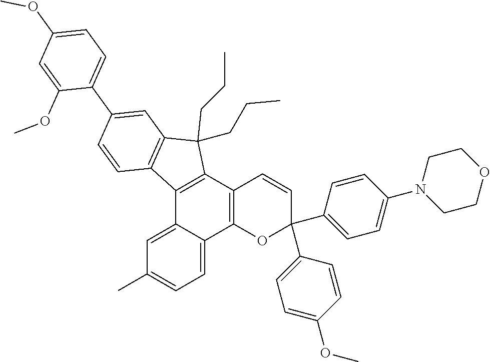 [SMILES: CCCC1(CCC)c2cc(-c3ccc(OC)cc3OC)ccc2-c2c1c1c(c3ccc(C)cc23)OC(c2ccc(OC)cc2)(c2ccc(N3CCOCC3)cc2)C=C1] | 5 | 86.8 | −2.6 | 2.7 | 6.6 | 12.1 | −10.9 | −23.6 | 24.4 | 43 |
| 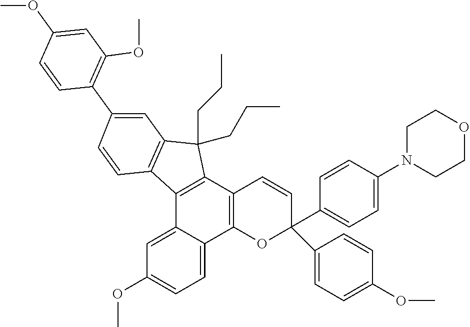 [SMILES: CCCC1(CCC)c2cc(-c3ccc(OC)cc3OC)ccc2-c2c1c1c(c3ccc(OC)cc23)OC(c2ccc(OC)cc2)(c2ccc(N3CCOCC3)cc2)C=C1] | CE5 | 83.2 | −2.0 | 1.4 | 9.4 | 8.6 | −5.1 | −17.0 | 18.4 | 57 |
| 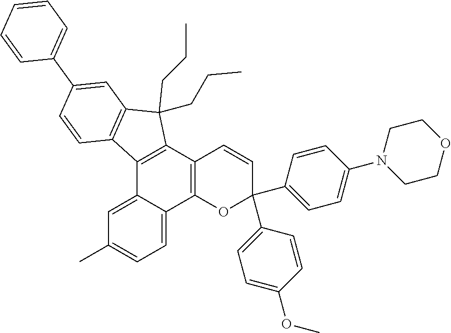 [SMILES: CCCC1(CCC)c2cc(-c3ccccc3)ccc2-c2c1c1c(c3ccc(C)cc23)OC(c2ccc(OC)cc2)(c2ccc(N3CCOCC3)cc2)C=C1] | 6 | 88.0 | −1.9 | 2.0 | 5.0 | 13.3 | −5.8 | −26.4 | 27.9 | 32 |
| 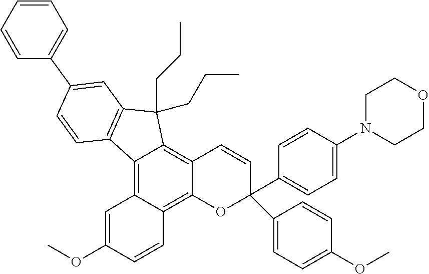 [SMILES: CCCC1(CCC)c2cc(-c3ccccc3)ccc2-c2c1c1c(c3ccc(OC)cc23)OC(c2ccc(OC)cc2)(c2ccc(N3CCOCC3)cc2)C=C1] | CE6 | 83.8 | −1.6 | 1.1 | 8.7 | 9.2 | 0.0 | −20.1 | 21.0 | 43 |
| 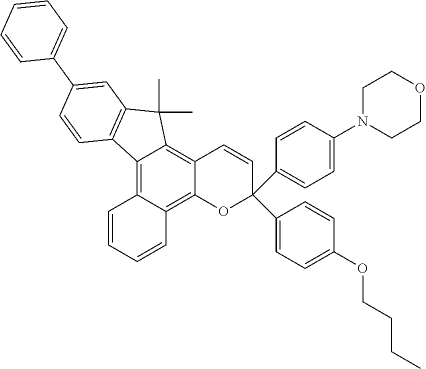 [SMILES: CCCCOc1ccc(C2(c3ccc(N4CCOCC4)cc3)C=Cc3c4c(c5ccccc5c3O2)-c2ccc(-c3ccccc3)cc2C4(C)C)cc1] | CE6A | 89.6 | −1.7 | 2.8 | 4.0 | 30.3 | −8.6 | −27.4 | 36.7 | 30 |
| 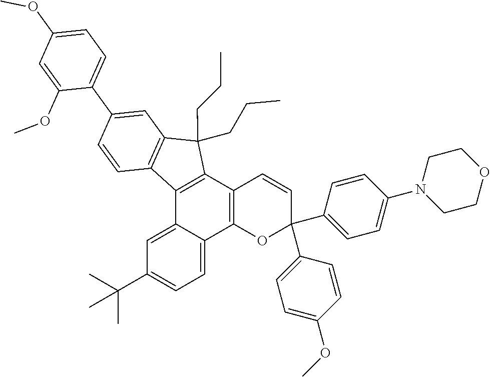 [SMILES: CCCC1(CCC)c2cc(-c3ccc(OC)cc3OC)ccc2-c2c1c1c(c3ccc(C(C)(C)C)cc23)OC(c2ccc(OC)cc2)(c2ccc(N3CCOCC3)cc2)C=C1] | 11 | 87.9 | −2.5 | 3.0 | 5.7 | 13.6 | −11.9 | −22.5 | 27.6 | 37 |
| 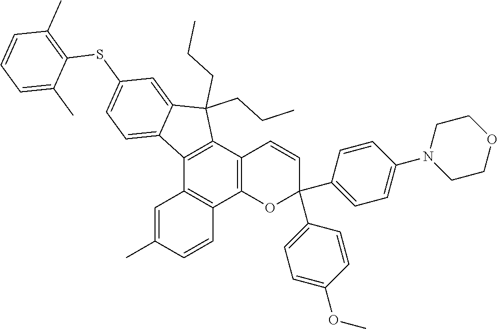 [SMILES: CCCC1(CCC)c2cc(Sc3c(C)cccc3C)ccc2-c2c1c1c(c3ccc(C)cc23)OC(c2ccc(OC)cc2)(c2ccc(N3CCOCC3)cc2)C=C1] | 7 | 87.5 | −2.3 | 2.3 | 5.7 | 13.7 | −11.5 | −23.8 | 28.0 | 38 |
| 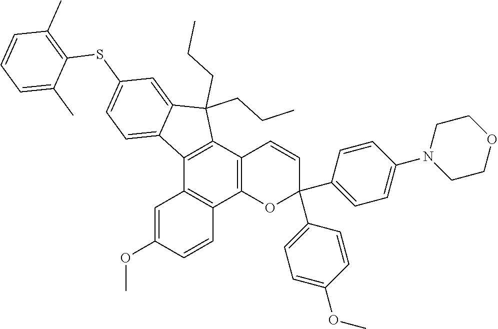 [SMILES: CCCC1(CCC)c2cc(Sc3c(C)cccc3C)ccc2-c2c1c1c(c3ccc(OC)cc23)OC(c2ccc(OC)cc2)(c2ccc(N3CCOCC3)cc2)C=C1] | CE7 | 84.5 | −1.8 | 1.2 | 8.1 | 10.1 | −6.0 | −18.1 | 22.4 | 47 |
| 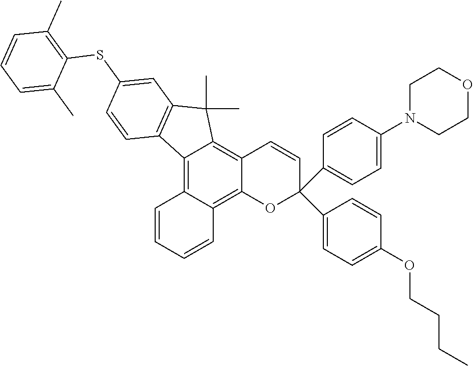 [SMILES: CCCCOc1ccc(C2(c3ccc(N4CCOCC4)cc3)C=Cc3c4c(c5ccccc5c3O2)-c2ccc(Sc3c(C)cccc3C)cc2C4(C)C)cc1] | CE7A | 88.6 | −2.8 | 3.4 | 5.5 | 17.6 | −14.5 | −26.0 | 32.9 | 34 |
| 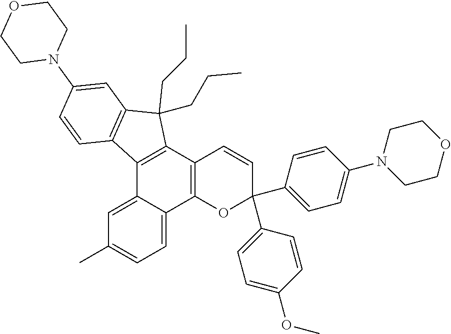 [SMILES: CCCC1(CCC)c2cc(N3CCOCC3)ccc2-c2c1c1c(c3ccc(C)cc23)OC(c2ccc(OC)cc2)(c2ccc(N3CCOCC3)cc2)C=C1] | 8 | 86.3 | −7.1 | 10.1 | 13.5 | 9.8 | −36.3 | −6.4 | 18.3 | 81 |
| 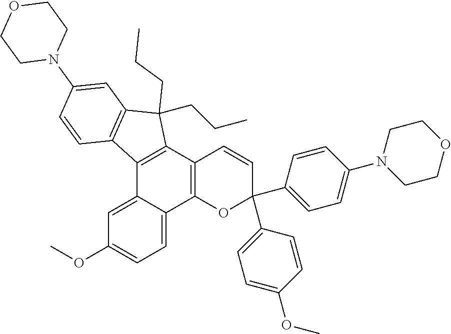 [SMILES: CCCC1(CCC)c2cc(N3CCOCC3)ccc2-c2c1c1c(c3ccc(OC)cc23)OC(c2ccc(OC)cc2)(c2ccc(N3CCOCC3)cc2)C=C1] | CE8 | 82.2 | −5.4 | 6.6 | 13.1 | 7.9 | −28.9 | −3.1 | 15.6 | 115 |
| 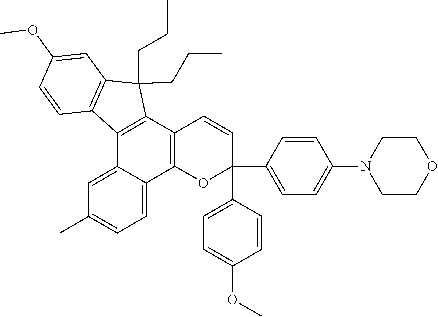 [SMILES: CCCC1(CCC)c2cc(OC)ccc2-c2c1c1c(c3ccc(C)cc23)OC(c2ccc(OC)cc2)(c2ccc(N3CCOCC3)cc2)C=C1] | 9 | 86.9 | −2.0 | 2.0 | 6.1 | 10.9 | −9.8 | −24.4 | 22.0 | 61 |
| 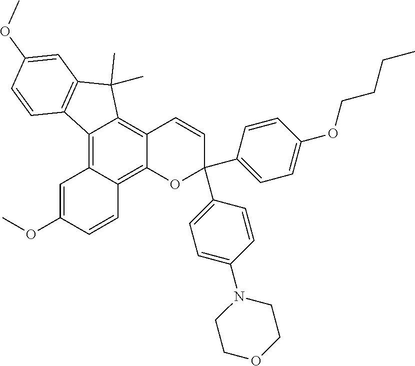 [SMILES: CCCCOc1ccc(C2(c3ccc(N4CCOCC4)cc3)C=Cc3c4c(c5cc(OC)ccc5c3O2)-c2ccc(OC)cc2C4(C)C)cc1] | CE9 | 82.0 | −1.7 | 0.5 | 10.4 | 6.1 | −4.1 | −19.0 | 13.9 | 121 |
| 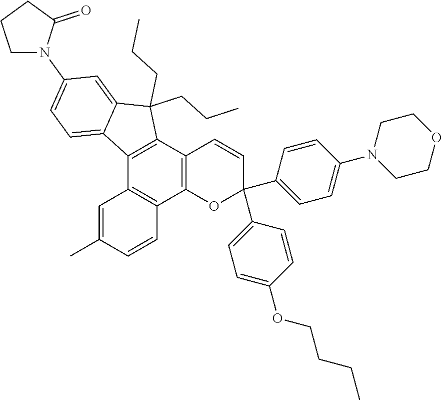 [SMILES: CCCCOc1ccc(C2(c3ccc(N4CCOCC4)cc3)C=Cc3c4c(c5cc(C)ccc5c3O2)-c2ccc(N3CCCC3=O)cc2C4(CCC)CCC)cc1] | 10 | 87.7 | −2.3 | 1.9 | 5.3 | 12.2 | −12.1 | −24.7 | 25.4 | 49 |
| 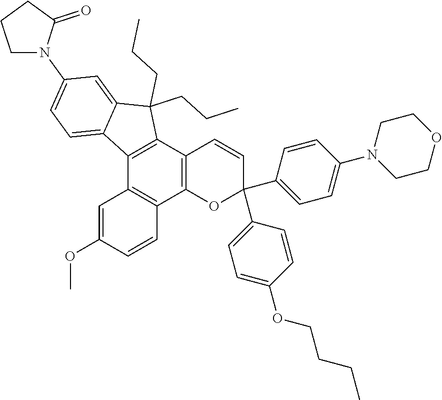 [SMILES: CCCCOc1ccc(C2(c3ccc(N4CCOCC4)cc3)C=Cc3c4c(c5cc(OC)ccc5c3O2)-c2ccc(N3CCCC3=O)cc2C4(CCC)CCC)cc1] | CE10 | 83.4 | −1.9 | 1.1 | 9.2 | 10.1 | −5.8 | −17.9 | 20.9 | 61 |

The results shown in Table A above clearly demonstrate the improvement in Delta E_{% T} bleach color through the use of the photochromic indeno-fused naphthopyran compounds of the present invention. These lower Delta E_{% T} values correspond to a photochromic article with good indoor clarity for improved visual acuity in lower light conditions and desirable aesthetics with the 7-position alkyl substituent. The 7-position alkyl groups also show an improvement in the activated darkness over the compounds having no substitution at the 7-position. Such is necessary to provide comfort and protection when wearing the lenses outdoors. The unexpected combination of excellent bleach color and darkening properties make the indeno-fused naphthopyran compounds of the present invention with the 7-position alkyl substituent ideal candidates for use in photochromatic lenses.

The present invention has been described with reference to specific details of particular embodiments thereof. It is not intended that such details be regarded as limitations upon the scope of the invention except insofar as and to the extent that they are included in the accompanying claims.

1. An indeno-fused naphthopyran having the following core skeletal structure (I): 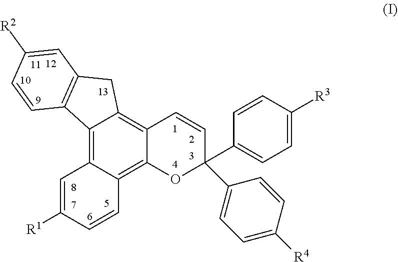
wherein:
R¹ is a substituted or unsubstituted alkyl group;
R² is substituted or unsubstituted amino;
substituted or unsubstituted alkyl;
substituted or unsubstituted alkenyl;
substituted or unsubstituted alkynyl;
substituted or unsubstituted aryl;
substituted or unsubstituted heteroaryl;
substituted or unsubstituted alkoxy;
substituted or unsubstituted aryloxy;
substituted or unsubstituted alkylthio;
substituted or unsubstituted arylthio;
substituted or unsubstituted amide;
substituted or unsubstituted urea; or
substituted or unsubstituted carbamate; and
at least one of R³ and R⁴ is an electron donating group, wherein the sum of the Hammett σₚ values for R³ and R⁴ is less than −0.40,
wherein when R¹ is a substituted alkyl group, a carbon atom of the alkyl group is directly bonded to the 7-position carbon atom, and the carbon atom of the alkyl group that is directly bonded to the 7-position carbon atom is unsubstituted.

2. The indeno-fused naphthopyran of claim 1, wherein R¹ is a C₁ to C₂₀ substituted or unsubstituted alkyl group.

3. The indeno-fused naphthopyran of claim 1, wherein R¹ is an unsubstituted C₁ to C₆ alkyl group.

4. The indeno-fused naphthopyran of claim 1, wherein R² is selected from the group consisting of substituted or unsubstituted aryl, substituted or unsubstituted amino, substituted or unsubstituted alkyoxy, substituted or unsubstituted aryloxy, substituted or unsubstituted alkylthio, and substituted or unsubstituted arylthiol.

5. The indeno-fused naphthopyran of claim 1, wherein R² is substituted or unsubstituted aryl.

6. The indeno-fused naphthopyran of claim 1, wherein at least one of R³ and R⁴ is a substituted or unsubstituted amino group.

7. The indeno-fused naphthopyran of claim 1, wherein at least one of R³ and R⁴ is a substituted or unsubstituted cyclic amino group.

8. The indeno-fused naphthopyran of claim 7, wherein at least one of R3 and R4 is a cyclic amino group which is a nitrogen-containing heterocycle selected from the group consisting of morpholino, piperidino, substituted piperazino, and pyrrolidino.

9. The indeno-fused naphthopyran of claim 1, wherein R³ and R⁴ are the same or different, and each is independently substituted or unsubstituted alkoxy or substituted or unsubstituted amino.

10. The indeno-fused naphthopyran of claim 1, wherein at least one of R³ and R⁴ is selected from the group consisting of methoxy, morpholino, piperazino, substituted piperazino, and dialkyl (C₁ to C₆) amino.

11. The indeno-naphthopyran of claim 1, having the following core skeletal structure (Ia); 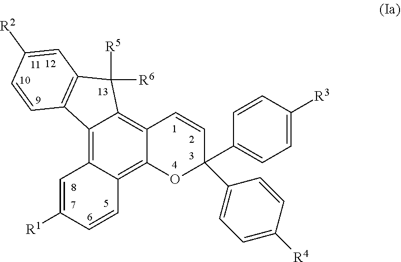
wherein:
R¹, R², R³, and R⁴ are each as described above with respect to core skeletal structure (I); and
R⁵ and R⁶ are each independently:
(i) hydrogen, substituted or unsubstituted alkyl, substituted or unsubstituted heterocycloalkyl, allyl, substituted or unsubstituted aryl, or substituted or unsubstituted heteroaryl;
(ii) alkoxy, hydroxyl, alkylthio, ketone, aldehyde, ester, carboxylic acid, carboxylate, siloxane, alkoxysilane, or polysiloxane;
(iii) a group comprising polyester, polyether, polycarbonate, polyurethane or combinations thereof; or
(iv) R⁵ and R⁶ together form an aliphatic ring having 3 to 20 ring member carbon atoms, a condensed polycyclic ring having an aromatic ring or aromatic hetero ring condensed to the above aliphatic ring, a hetero ring having 3 to 20 ring member atoms, or a condensed polycyclic ring having an aromatic ring or aromatic hetero ring condensed to the above hetero ring, together with the 13-position carbon atom bonded thereto.

12. The indeno-fused naphthopyran of claim 11, wherein R⁵ and R⁶ are each independently substituted or unsubstituted alkyl.

13. The indeno-fused naphthopyran of claim 11, wherein
R¹ is unsubstituted C₁ to C₆ alkyl;
R² is substituted or unsubstituted phenyl;
R³ and R⁴ are each independently C₁ to C₄ alkoxy or a nitrogen-containing heterocycle; and
R⁵ and R⁶ are each independently C₁ to C₄ alkyl.

14. A photochromic composition comprising the indeno-fused naphthopyran of claim 1.

15. A photochromic article comprising the indeno-fused naphthopyran of claim 1,
wherein the photochromic article is selected from ophthalmic articles, display articles, windows, mirrors, active liquid crystal cell articles, or passive liquid crystal cell articles; or
wherein the photochromic article is selected from ophthalmic articles, and the ophthalmic articles are selected from corrective lenses, non-corrective lenses, contact lenses, intra-ocular lenses, magnifying lenses, protective lenses, or visors; or
wherein the photochromic article is selected from display articles, and the display articles are selected from screens, monitors, or security elements.
